(19) United States
(12) Patent Application Publication
(10) Pub. No.: US 2008/0113946 A1
(43) Pub. Date: May 15, 2008

(54) N-aryl-5,7-dihydrofuro[3,4-d]pyrimidin-4-amines and analogs as activators of caspases and inducers of apoptosis and the use thereof

(75) Inventors: Han-Zhong Zhang, San Diego, CA (US); Sui Xiong Cai, San Diego, CA (US); William Edward Kemnitzer, San Diego, CA (US)

Correspondence Address: STERNE, KESSLER, GOLDSTEIN & FOX P.L.L.C., 1100 NEW YORK AVENUE, N.W., WASHINGTON, DC, 20005, US

(73) Assignee: Cyntovia, Inc., San Diego, CA (US)

(21) Appl. No.: 11/984,179

(22) Filed: Nov. 14, 2007

Related U.S. Application Data
(63) Continuation-in-part of application No. PCT/US07/18183, filed on Aug. 16, 2007.
(60) Provisional application No. 60/837,921, filed on Aug. 16, 2006.
(60) Provisional application No. 60/858,695, filed on Nov. 14, 2006.

(51) Int. Cl. A61K 31/655; A61K 31/519

(52) U.S. Cl. 514/151; 514/260.1; 514/265.1; 514/258.1

(57) ABSTRACT

Disclosed are N-aryl-5,7-dihydrofuro[3,4-d]pyrimidin-4-amines and analogs thereof, represented by the Formula I: 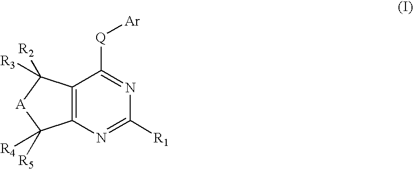
wherein Ar, A, Q and R₁-R₆ are defined herein. The present invention relates to the discovery that compounds having Formula I are activators of caspases and inducers of apoptosis. Therefore, the activators of caspases and inducers of apoptosis of this invention may be used to induce cell death in a variety of clinical conditions in which uncontrolled growth and spread of abnormal cells occurs.

### BACKGROUND OF THE INVENTION

1. Field of the Invention

This invention is in the field of medicinal chemistry. In particular, the invention relates to N-aryl-5,7-dihydrofuro[3,4-d]pyrimidin-4-amines and analogs, and the discovery that these compounds are activators of caspases and inducers of apoptosis. The invention also relates to the use of these compounds as therapeutically effective anti-cancer agents.

2. Related Art

Organisms eliminate unwanted cells by a process variously known as regulated cell death, programmed cell death or apoptosis. Such cell death occurs as a normal aspect of animal development, as well as in tissue homeostasis and aging (Glucksmann, A., Biol. Rev. Cambridge Philos. Soc. 26:59-86 (1951); Glucksmann, A., Archives de Biologie 76:419-437 (1965); Ellis, et al., Dev. 112:591-603 (1991); Vaux, et al., Cell 76:777-779 (1994)). Apoptosis regulates cell number, facilitates morphogenesis, removes harmful or otherwise abnormal cells and eliminates cells that have already performed their function. Additionally, apoptosis occurs in response to various physiological stresses, such as hypoxia or ischemia (PCT published application WO96/20721).

There are a number of morphological changes shared by cells experiencing regulated cell death, including plasma and nuclear membrane blebbing, cell shrinkage (condensation of nucleoplasm and cytoplasm), organelle relocalization and compaction, chromatin condensation and production of apoptotic bodies (membrane enclosed particles containing intracellular material) (Orrenius, S., J. Internal Medicine 237:529-536 (1995)).

Apoptosis is achieved through an endogenous mechanism of cellular suicide (Wyllie, A. H., in Cell Death in Biology and Pathology, Bowen and Lockshin, eds., Chapman and Hall (1981), pp. 9-34). A cell activates its internally encoded suicide program as a result of either internal or external signals. The suicide program is executed through the activation of a carefully regulated genetic program (Wyllie, et al., Int. Rev. Cyt. 68:251 (1980); Ellis, et al., Ann. Rev. Cell Bio. 7:663 (1991)). Apoptotic cells and bodies are usually recognized and cleared by neighboring cells or macrophages before lysis. Because of this clearance mechanism, inflammation is not induced despite the clearance of great numbers of cells (Orrenius, S., J. Internal Medicine 237:529-536 (1995)).

It has been found that a group of proteases are a key element in apoptosis (see, e.g., Thornberry, Chemistry and Biology 5:R97-R103 (1998); Thornberry, British Med. Bull. 53:478-490 (1996)). Genetic studies in the nematode Caenorhabditis elegans revealed that apoptotic cell death involves at least 14 genes, 2 of which are the pro-apoptotic (death-promoting) ced (for cell death abnormal) genes, ced-3 and ced-4. CED-3 is homologous to interleukin 1 beta-converting enzyme, a cysteine protease, which is now called caspase-1. When these data were ultimately applied to mammals, and upon further extensive investigation, it was found that the mammalian apoptosis system appears to involve a cascade of caspases, or a system that behaves like a cascade of caspases. At present, the caspase family of cysteine proteases comprises 14 different members, and more may be discovered in the future. All known caspases are synthesized as zymogens that require cleavage at an aspartyl residue prior to forming the active enzyme. Thus, caspases are capable of activating other caspases, in the manner of an amplifying cascade.

Apoptosis and caspases are thought to be crucial in the development of cancer (Apoptosis and Cancer Chemotherapy, Hickman and Dive, eds., Humana Press (1999)). There is mounting evidence that cancer cells, while containing caspases, lack parts of the molecular machinery that activates the caspase cascade. This makes the cancer cells lose their capacity to undergo cellular suicide and the cells become cancerous. In the case of the apoptosis process, control points are known to exist that represent points for intervention leading to activation. These control points include the CED-9-BCL-like and CED-3-ICE-like gene family products, which are intrinsic proteins regulating the decision of a cell to survive or die and executing part of the cell death process itself, respectively (see, Schmitt, et al., Biochem. Cell. Biol. 75:301-314 (1997)). BCL-like proteins include BCL-xL and BAX-alpha, which appear to function upstream of caspase activation. BCL-xL appears to prevent activation of the apoptotic protease cascade, whereas BAX-alpha accelerates activation of the apoptotic protease cascade.

It has been shown that chemotherapeutic (anti-cancer) drugs can trigger cancer cells to undergo suicide by activating the dormant caspase cascade. This may be a crucial aspect of the mode of action of most, if not all, known anticancer drugs (Los, et al., Blood 90:3118-3129 (1997); Friesen, et al., Nat. Med. 2:574 (1996)). The mechanism of action of current antineoplastic drugs frequently involves an attack at specific phases of the cell cycle. In brief, the cell cycle refers to the stages through which cells normally progress during their lifetime. Normally, cells exist in a resting phase termed Gₒ. During multiplication, cells progress to a stage in which DNA synthesis occurs, termed S. Later, cell division, or mitosis occurs, in a phase called M. Antineoplastic drugs, such as cytosine arabinoside, hydroxyurea, 6-mercaptopurine, and methotrexate are S phase specific, whereas antineoplastic drugs, such as vincristine, vinblastine, and paclitaxel are M phase specific. Many slow growing tumors, e.g. colon cancers, exist primarily in the Gₒ phase, whereas rapidly proliferating normal tissues, for example bone marrow, exist primarily in the S or M phase. Thus, a drug like 6-mercaptopurine can cause bone marrow toxicity while remaining ineffective for a slow growing tumor. Further aspects of the chemotherapy of neoplastic diseases are known to those skilled in the art (see, e.g., Hardman, et al., eds., Goodman and Gilman's The Pharmacological Basis of Therapeutics, Ninth Edition, McGraw-Hill, New York (1996), pp. 1225-1287). Thus, it is clear that the possibility exists for the activation of the caspase cascade, although the exact mechanisms for doing so are not clear at this point. It is equally clear that insufficient activity of the caspase cascade and consequent apoptotic events are implicated in various types of cancer. The development of caspase cascade activators and inducers of apoptosis is a highly desirable goal in the development of therapeutically effective antineoplastic agents. Moreover, since autoimmune disease and certain degenerative diseases also involve the proliferation of abnormal cells, therapeutic treatment for these diseases could also involve the enhancement of the apoptotic process through the administration of appropriate caspase cascade activators and inducers of apoptosis.

Pluta et al. (Acta Poloniae Pharmaceutica 51: 55-8 (1994)) reported antibacterial properties of some 2,7-dihydrofuro[3,4-d]-pyrimidines. Antibacterial screening data against Staphylococcus aureus, Proteus vulgaris, Pseudomonas aeruginosa and Escherichia coli were reported for 1 (R=H, 2-Cl, 4-Cl, 4-EtO, 4-Me, 4-OH) and II (R₁=E)- and (Z)-CH₂CH₂NEt₂, R₂=O; R₁=R₂=4-ClC₆H₄N, 3,5-C₁₂C₆H₃N, PrN, ClCH₂CH₂N).

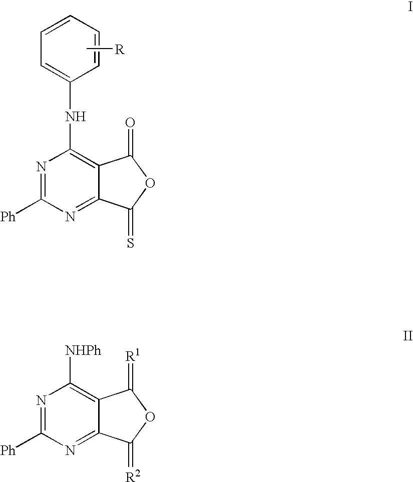

U.S. Pat. No. 4,749,704 disclosed cyclopenta[d]pyrimidine derivatives and their use as antidepressants.

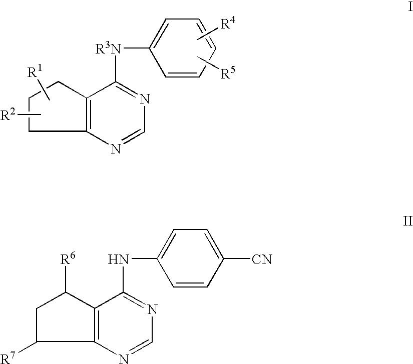

Compounds of formula I [R₁=OH, alkenyloxy, (un)substituted alkoxy, aryloxy, aliphatic or aromatic acyloxy; R₂=H, and R₁; R₃=H, alkyl; R₄, R₅=H, alkyl, alkoxy, OH, halo, NO₂, cyano, CO₂H, etc.] were prepared as antidepressants, with specific examples such as those of formula II.

Tome et al. (Tetrahedron 52: 1735-46 (1996)) reported that pyrimidine sulfones I (R=Me, Ph; R₁=OMe, SPh, NEt₂) were synthesized from the dihydrothienopyrimidones by conversion to their chloro derivatives, followed by oxidation with m-chloroperbenzoic (mCPBA) and reaction with the appropriate nucleophile.

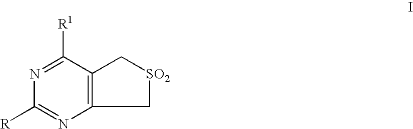

EP1552842 disclosed the preparation of bicyclic pyrimidine derivatives I as antiinflammatory agents for treatment of allergic diseases.

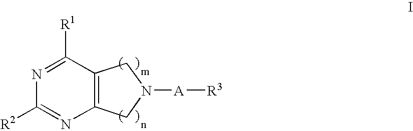

Compounds of formula I, wherein m and n=independently 1-3; R₁=(un)substituted amino; R₂=—B—(CX₂)p-R₇, (un)substituted piperidinyl, piperazinyl, or amino; B=O, CH=CH, or phenylene; p=1-4; X=H, halo, or (un)substituted alkyl; R₇=(un)substituted amino; A=a single bond, CO, SO₂, OCO, OCS, SCO, SCS, (un)substituted NHCO, NHCS, or amino; R₃=H, (un)substituted alkyl, cycloalkyl, alkenyl, alkynyl, aralkyl, aryl, heteroaryl, heterocyclyl, heteroarylalkyl, or heterocyclylalkyl, etc., were prepared.

### SUMMARY OF THE INVENTION

The present invention is related to the discovery that N-aryl-5,7-dihydrofuro[3,4-d]pyrimidin-4-amines and analogs, as represented in Formulae I-V, are activators of the caspase cascade and inducers of apoptosis. Thus, an aspect of the present invention is directed to the use of compounds of Formulae I-V as inducers of apoptosis.

A second aspect of the present invention is to provide a method for treating, preventing or ameliorating neoplasia and cancer by administering a compound of one of the Formulae I-V to a mammal in need of such treatment.

Many of the compounds within the scope of the present invention are novel compounds. Therefore, a third aspect of the present invention is to provide novel compounds of Formulae I-V, and to also provide for the use of these novel compounds for treating, preventing or ameliorating neoplasia and cancer.

A fourth aspect of the present invention is to provide a pharmaceutical composition useful for treating disorders responsive to the induction of apoptosis, containing an effective amount of a compound of one of the Formulae I-V in admixture with one or more pharmaceutically acceptable carriers or diluents.

A fifth aspect of the present invention is directed to methods for the preparation of novel compounds of Formulae I-V.

### DETAILED DESCRIPTION OF THE INVENTION

The present invention arises out of the discovery that N-aryl-5,7-dihydrofuro[3,4-d]pyrimidin-4-amines and analogs, as represented in Formulae I-V, are potent and highly efficacious activators of the caspase cascade and inducers of apoptosis. Therefore, compounds of Formulae I-V are useful for treating disorders responsive to induction of apoptosis.

Specifically, compounds of the present invention are represented by Formula I:

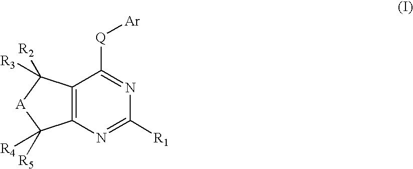

or pharmaceutically acceptable salts or prodrugs or tautomers thereof, wherein:

Ar is optionally substituted aryl or optionally substituted heteroaryl;

A is O, S(O)ₙ, NR₇, or CR₉R₁₀, wherein n is 0, 1 or 2; R₇ is R₈, COR₈, CONHR₈, COOR₈, or SO₂R₈, wherein R₈-R₁₀ independently are hydrogen, C₁₋₁₀ alkyl, haloalkyl, aryl, carbocyclic, a heterocyclic group, a heteroaryl group, alkenyl, alkynyl, arylalkyl, arylalkenyl, arylalkynyl, heteroarylalkyl, heteroarylalkenyl, heteroarylalkynyl, carbocycloalkyl, heterocycloalkyl, hydroxyalkyl, aminoalkyl, or carboxyalkyl;

Q is O, S or NR₆, wherein R₆ is hydrogen or an optionally substituted alkyl;

R₁ is hydrogen, halo, optionally substituted amino, alkylamino, arylamino, alkylthiol, alkylsulfone, alkylsulfoxide, arylthiol, alkoxy, or aryloxy, optionally substituted C₁₋₁₀ alkyl, haloalkyl, aryl, carbocyclic, a heterocyclic group, a heteroaryl group, alkenyl, alkynyl, arylalkyl, arylalkenyl, arylalkynyl, heteroarylalkyl, heteroarylalkenyl, heteroarylalkynyl, carbocycloalkyl, heterocycloalkyl, hydroxyalkyl, aminoalkyl, aminoaryl, carboxyalkyl, nitro, cyano, acylamido, hydroxy, acyloxy, azido, carboxy, or carbonylamido; and

R₂-R₅ independently are hydrogen, halo, amino, alkoxy, C₁₋₁₀ alkyl, haloalkyl, aryl, carbocyclic, a heterocyclic group, a heteroaryl group, alkenyl, alkynyl, arylalkyl, arylalkenyl, arylalkynyl, heteroarylalkyl, heteroarylalkenyl, heteroarylalkynyl, carbocycloalkyl, heterocycloalkyl, hydroxyalkyl, aminoalkyl, carboxyalkyl, nitro, cyano, acylamido, hydroxy, thiol, acyloxy, azido, carboxy, methylenedioxy, carbonylamido or alkylthiol, alkylsulfone, alkylsulfoxide.

Preferred compounds of Formula I include compounds wherein Ar is optionally substituted pyrrazol, triaziol, thiadiazol, imidazole, oxazol, thiazol, phenyl, pyridyl, or pyrimidyl. Another group of preferred compounds include compounds wherein Q is NH.

One group of compounds of the present invention are represented by Formula II:

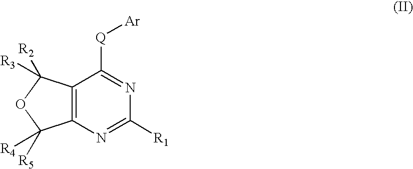

or pharmaceutically acceptable salts or prodrugs or tautomers thereof, wherein:

Ar is optionally substituted aryl or optionally substituted heteroaryl;

Q is O, S or NR₆, wherein R₆ is hydrogen or an optionally substituted alkyl;

R₁ is hydrogen, halo, optionally substituted amino, alkylamino, arylamino, alkylthiol, alkylsulfone, alkylsulfoxide, arylthiol, alkoxy, or aryloxy, optionally substituted C₁₋₁₀ alkyl, haloalkyl, aryl, carbocyclic, a heterocyclic group, a heteroaryl group, alkenyl, alkynyl, arylalkyl, arylalkenyl, arylalkynyl, heteroarylalkyl, heteroarylalkenyl, heteroarylalkynyl, carbocycloalkyl, heterocycloalkyl, hydroxyalkyl, aminoalkyl, aminoaryl, carboxyalkyl, nitro, cyano, acylamido, hydroxy, acyloxy, azido, carboxy, or carbonylamido; and

R₂-R₅ independently are hydrogen, halo, amino, alkoxy, C₁₋₁₀ alkyl, haloalkyl, aryl, carbocyclic, a heterocyclic group, a heteroaryl group, alkenyl, alkynyl, arylalkyl, arylalkenyl, arylalkynyl, heteroarylalkyl, heteroarylalkenyl, heteroarylalkynyl, carbocycloalkyl, heterocycloalkyl, hydroxyalkyl, aminoalkyl, carboxyalkyl, nitro, cyano, acylamido, hydroxy, thiol, acyloxy, azido, carboxy, methylenedioxy, carbonylamido or alkylthiol, alkylsulfone, alkylsulfoxide.

Preferred compounds of Formula II include compounds wherein Ar is optionally substituted pyrrazol, triaziol, thiadiazol, imidazole, oxazol, thiazol, phenyl, pyridyl, or pyrimidyl.

Another group of preferred compounds of the present invention are represented by Formula III:

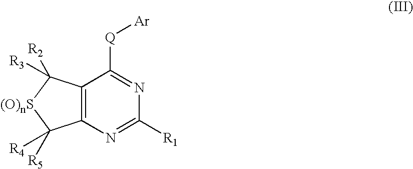

or pharmaceutically acceptable salts, prodrugs or tautomers thereof, wherein:

Ar is optionally substituted aryl or optionally substituted heteroaryl;

Q is O, S or NR₆, wherein R₆ is hydrogen or an optionally substituted alkyl;

n is 0, 1 or 2.

R₁ is hydrogen, halo, optionally substituted amino, alkylamino, arylamino, alkylthiol, alkylsulfone, alkylsulfoxide, arylthiol, alkoxy, or aryloxy, optionally substituted C₁₋₁₀ alkyl, haloalkyl, aryl, carbocyclic, a heterocyclic group, a heteroaryl group, alkenyl, alkynyl, arylalkyl, arylalkenyl, arylalkynyl, heteroarylalkyl, heteroarylalkenyl, heteroarylalkynyl, carbocycloalkyl, heterocycloalkyl, hydroxyalkyl, aminoalkyl, aminoaryl, carboxyalkyl, nitro, cyano, acylamido, hydroxy, acyloxy, azido, carboxy, or carbonylamido; and

R₂-R₅ independently are hydrogen, halo, amino, alkoxy, C₁₋₁₀ alkyl, haloalkyl, aryl, carbocyclic, a heterocyclic group, a heteroaryl group, alkenyl, alkynyl, arylalkyl, arylalkenyl, arylalkynyl, heteroarylalkyl, heteroarylalkenyl, heteroarylalkynyl, carbocycloalkyl, heterocycloalkyl, hydroxyalkyl, aminoalkyl, carboxyalkyl, nitro, cyano, acylamido, hydroxy, thiol, acyloxy, azido, carboxy, methylenedioxy, carbonylamido or alkylthiol, alkylsulfone, alkylsulfoxide

Preferred compounds of Formula III include compounds wherein Ar is optionally substituted pyrrazol, triaziol, thiadiazol, imidazole, oxazol, thiazol, phenyl, pyridyl, or pyrimidyl.

Another group of preferred compounds of the present invention are represented by Formula IV:

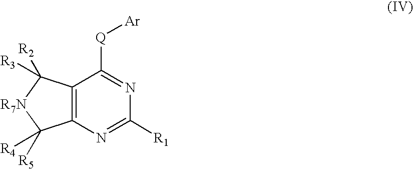

or pharmaceutically acceptable salts, prodrugs or tautomers thereof, wherein:

Ar is optionally substituted aryl or optionally substituted heteroaryl;

Q is O, S or NR₆, wherein R₆ is hydrogen or an optionally substituted alkyl;

R₁ is hydrogen, halo, optionally substituted amino, alkylamino, arylamino, alkylthiol, alkylsulfone, alkylsulfoxide, arylthiol, alkoxy, or aryloxy, optionally substituted C₁₋₁₀ alkyl, haloalkyl, aryl, carbocyclic, a heterocyclic group, a heteroaryl group, alkenyl, alkynyl, arylalkyl, arylalkenyl, arylalkynyl, heteroarylalkyl, heteroarylalkenyl, heteroarylalkynyl, carbocycloalkyl, heterocycloalkyl, hydroxyalkyl, aminoalkyl, aminoaryl, carboxyalkyl, nitro, cyano, acylamido, hydroxy, acyloxy, azido, carboxy, or carbonylamido;

R₂-R₅ independently are hydrogen, halo, amino, alkoxy, C₁₋₁₀ alkyl, haloalkyl, aryl, carbocyclic, a heterocyclic group, a heteroaryl group, alkenyl, alkynyl, arylalkyl, arylalkenyl, arylalkynyl, heteroarylalkyl, heteroarylalkenyl, heteroarylalkynyl, carbocycloalkyl, heterocycloalkyl, hydroxyalkyl, aminoalkyl, carboxyalkyl, nitro, cyano, acylamido, hydroxy, thiol, acyloxy, azido, carboxy, methylenedioxy, carbonylamido or alkylthiol, alkylsulfone, alkylsulfoxide; and

R₇ is R₈, COR₈, CONHR₈, COOR₈, or SO₂R₈, wherein R₈ is hydrogen, C₁₋₁₀ alkyl, haloalkyl, aryl, carbocyclic, a heterocyclic group, a heteroaryl group, alkenyl, alkynyl, arylalkyl, arylalkenyl, arylalkynyl, heteroarylalkyl, heteroarylalkenyl, heteroarylalkynyl, carbocycloalkyl, heterocycloalkyl, hydroxyalkyl, aminoalkyl, or carboxyalkyl;

Preferred compounds of Formula IV include compounds wherein Ar is optionally substituted pyrrazol, triaziol, thiadiazol, imidazole, oxazol, thiazol, phenyl, pyridyl, or pyrimidyl.

Another group of preferred compounds of the present invention are represented by Formula V:

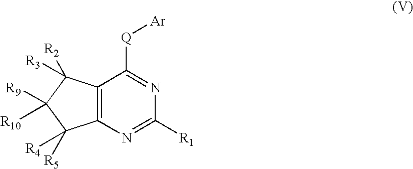

or pharmaceutically acceptable salts, prodrugs or tautomers thereof, wherein:

Ar is optionally substituted aryl or optionally substituted heteroaryl;

Q is O, S or NR₆, wherein R₆ is hydrogen or an optionally substituted alkyl;

R₁ is hydrogen, halo, optionally substituted amino, alkylamino, arylamino, alkylthiol, alkylsulfone, alkylsulfoxide, arylthiol, alkoxy, or aryloxy, optionally substituted C₁₋₁₀ alkyl, haloalkyl, aryl, carbocyclic, a heterocyclic group, a heteroaryl group, alkenyl, alkynyl, arylalkyl, arylalkenyl, arylalkynyl, heteroarylalkyl, heteroarylalkenyl, heteroarylalkynyl, carbocycloalkyl, heterocycloalkyl, hydroxyalkyl, aminoalkyl, aminoaryl, carboxyalkyl, nitro, cyano, acylamido, hydroxy, acyloxy, azido, carboxy, or carbonylamido;

R₂-R₅ and R₉-R₁₀ independently are hydrogen, halo, amino, alkoxy, C₁₋₁₀ alkyl, haloalkyl, aryl, carbocyclic, a heterocyclic group, a heteroaryl group, alkenyl, alkynyl, arylalkyl, arylalkenyl, arylalkynyl, heteroarylalkyl, heteroarylalkenyl, heteroarylalkynyl, carbocycloalkyl, heterocycloalkyl, hydroxyalkyl, aminoalkyl, carboxyalkyl, nitro, cyano, acylamido, hydroxy, thiol, acyloxy, azido, carboxy, methylenedioxy, carbonylamido or alkylthiol, alkylsulfone, alkylsulfoxide; and

Preferred compounds of Formula V include compounds wherein Ar is optionally substituted pyrrazol, triaziol, thiadiazol, imidazole, oxazol, thiazol, phenyl, pyridyl, or pyrimidyl.

Exemplary preferred compounds of Formulae I-V that may be employed in the method of the invention include, without limitation:
5,7-Dihydro-N-(3,5-dimethoxyphenyl)-2-(methylthio)furo[3,4-d]pyrimidin-4-amine;
5,7-Dihydro-N-(4-methoxyphenyl)-N-methyl-2-(methylthio)furo[3,4-d]pyrimidin-4-amine;
5,7-Dihydro-N-(3,5-dimethoxyphenyl)-furo[3,4-d]pyrimidin-4-amine;
5,7-Dihydro-N-(3-bromophenyl)-2-methylfuro[3,4-d]pyrimidin-4-amine;
5,7-Dihydro-N-(3,5-dimethoxyphenyl)-2-methylfuro[3,4-d]pyrimidin-4-amine;
5,7-Dihydro-N-(3-methyl-1H-pyrazol-5-yl)-2-(methylthio)furo[3,4-d]pyrimidin-4-amine;
5,7-Dihydro-N-(3-methyl-1H-pyrazol-5-yl)-2-(methylsulfonyl)furo[3,4-d]pyrimidin-4-amine;
N-(4-(4-(3-Methyl-1H-pyrazol-5-ylamino)-5,7-dihydrofuro[3,4-d]pyrimidin-2-ylthio)phenyl)cyclopropanecarboxamide;
N-(4-(5,7-Dihydro-2-(methylsulfonyl)furo[3,4-d]pyrimidin-4-ylthio)phenyl)cyclopropanecarboxamide;
N-(4-(2-(3-Methyl-1H-pyrazol-5-ylamino)-5,7-dihydrofuro[3,4-d]pyrimidin-4-ylthio)phenyl)cyclopropanecarboxamide; and
(9H-Fluoren-9-yl)methyl 4-(3-methyl-1H-pyrazol-5-ylamino)-2-chloro-5H-pyrrolo[3,4-d]pyrimidine-6(7H)-carboxylate;
6,7-Dihydro-N-(2,5-dimethoxyphenyl)-2-(methylthio)-5H-cyclopenta[d]pyrimidin-4-amine;
N-(3-Bromophenyl)-6,7-dihydro-2-(methylthio)-5H-cyclopenta[d]pyrimidin-4-amine;
6,7-Dihydro-N-(5-methyl-1H-pyrazol-3-yl)-2-(methylthio)-5H-cyclopenta[d]pyrimidin-4-amine;
6,7-Dihydro-N-(3,5-dimethoxyphenyl)-2-(methylthio)-5H-cyclopenta[d]pyrimidin-4-amine;
N-(4-(6,7-Dihydro-2-(methylthio)-5H-cyclopenta[d]pyrimidin-4-ylamino)phenyl)benzamide;
6,7-Dihydro-N-(4-methoxyphenyl)-N-methyl-2-(methylthio)-5H-cyclopenta[d]pyrimidin-4-amine;
6,7-Dihydro-N-(3,5-dimethoxyphenyl)-5H-cyclopenta[d]pyrimidin-4-amine;
6,7-Dihydro-N-(2,5-dimethoxyphenyl)-5H-cyclopenta[d]pyrimidin-4-amine;
N-(4-(6,7-Dihydro-5H-cyclopenta[d]pyrimidin-4-ylamino)phenyl)benzamide;
6,7-Dihydro-N-(3,5-dimethoxyphenyl)-2-(methylsulfinyl)-5H-cyclopenta[d]pyrimidin-4-amine;
(9H-Fluoren-9-yl)methyl 4-(3-(dimethylamino)-5-methoxyphenylamino)-2-chloro-5H-pyrrolo[3,4-d]pyrimidine-6(7H)-carboxylate;
(9H-Fluoren-9-yl)methyl 4-(N-(4-methoxyphenyl)-N-methylamino)-2-chloro-5H-pyrrolo[3,4-d]pyrimidine-6(7H)-carboxylate; and
5,7-Dihydro-N-(3-methyl-1H-pyrazol-5-yl)-2-(4-methylpiperazin-1-yl)furo[3,4-d]pyrimidin-4-amine;

and pharmaceutically acceptable salts or prodrugs thereof

The present invention is also directed to novel compounds within the scope of Formulae I-V. Exemplary preferred compounds that may be employed in this invention include, without limitation:
5,7-Dihydro-N-(3,5-dimethoxyphenyl)-2-(methylthio)furo[3,4-d]pyrimidin-4-amine;
5,7-Dihydro-N-(4-methoxyphenyl)-N-methyl-2-(methylthio)furo[3,4-d]pyrimidin-4-amine;
5,7-Dihydro-N-(3,5-dimethoxyphenyl)-furo[3,4-d]pyrimidin-4-amine;
5,7-Dihydro-N-(3-bromophenyl)-2-methylfuro[3,4-d]pyrimidin-4-amine;
5,7-Dihydro-N-(3,5-dimethoxyphenyl)-2-methylfuro[3,4-d]pyrimidin-4-amine;
5,7-Dihydro-N-(3-methyl-1H-pyrazol-5-yl)-2-(methylthio)furo[3,4-d]pyrimidin-4-amine;
5,7-Dihydro-N-(3-methyl-1H-pyrazol-5-yl)-2-(methylsulfonyl)furo[3,4-d]pyrimidin-4-amine;
N-(4-(4-(3-Methyl-1H-pyrazol-5-ylamino)-5,7-dihydrofuro[3,4-d]pyrimidin-2-ylthio)phenyl)cyclopropanecarboxamide;
N-(4-(5,7-Dihydro-2-(methylsulfonyl)furo[3,4-d]pyrimidin-4-ylthio)phenyl)cyclopropanecarboxamide;
N-(4-(2-(3-Methyl-1H-pyrazol-5-ylamino)-5,7-dihydrofuro[3,4-d]pyrimidin-4-ylthio)phenyl)cyclopropanecarboxamide;
(9H-Fluoren-9-yl)methyl 4-(3-methyl-1H-pyrazol-5-ylamino)-2-chloro-5H-pyrrolo[3,4-d]pyrimidine-6(7H)-carboxylate;
6,7-Dihydro-N-(2,5-dimethoxyphenyl)-2-(methylthio)-5H-cyclopenta[d]pyrimidin-4-amine;
N-(3-Bromophenyl)-6,7-dihydro-2-(methylthio)-5H-cyclopenta[d]pyrimidin-4-amine;
6,7-Dihydro-N-(5-methyl-1H-pyrazol-3-yl)-2-(methylthio)-5H-cyclopenta[d]pyrimidin-4-amine;
6,7-Dihydro-N-(3,5-dimethoxyphenyl)-2-(methylthio)-5H-cyclopenta[d]pyrimidin-4-amine;
N-(4-(6,7-Dihydro-2-(methylthio)-5H-cyclopenta[d]pyrimidin-4-ylamino)phenyl)benzamide;
6,7-Dihydro-N-(4-methoxyphenyl)-N-methyl-2-(methylthio)-5H-cyclopenta[d]pyrimidin-4-amine;
6,7-Dihydro-N-(3,5-dimethoxyphenyl)-5H-cyclopenta[d]pyrimidin-4-amine;
6,7-Dihydro-N-(2,5-dimethoxyphenyl)-5H-cyclopenta[d]pyrimidin-4-amine;
N-(4-(6,7-Dihydro)-5H-cyclopenta[d]pyrimidin-4-ylamino)phenyl)benzamide;
6,7-Dihydro-N-(3,5-dimethoxyphenyl)-2-(methylsulfinyl)-5H-cyclopenta[d]pyrimidin-4-amine;
(9H-Fluoren-9-yl)methyl 4-(3-(dimethylamino)-5-methoxyphenylamino)-2-chloro-5H-pyrrolo[3,4-d]pyrimidine-6(7H)-carboxylate;
(9H-Fluoren-9-yl)methyl 4-(N-(4-methoxyphenyl)-N-methylamino)-2-chloro-5H-pyrrolo[3,4-d]pyrimidine-6(7H)-carboxylate; and
5,7-Dihydro-N-(3-methyl-1H-pyrazol-5-yl)-2-(4-methylpiperazin-1-yl)furo[3,4-d]pyrimidin-4-amine;
   and pharmaceutically acceptable salts or prodrugs thereof.

The term “alkyl” as employed herein by itself or as part of another group refers to both straight and branched chain radicals of up to ten carbons. Useful alkyl groups include straight-chained and branched C₁₋₁₀ alkyl groups, more preferably C₁₋₆ alkyl groups. Typical C₁₋₁₀ alkyl groups include methyl, ethyl, propyl, isopropyl, butyl, sec-butyl, tert-butyl, 3-pentyl, hexyl and octyl groups, which may be optionally substituted.

The term “alkenyl” as employed herein by itself or as part of another group means a straight or branched chain radical of 2-10 carbon atoms, unless the chain length is limited thereto, including at least one double bond between two of the carbon atoms in the chain. Typical alkenyl groups include ethenyl, 1-propenyl, 2-propenyl, 2-methyl-1-propenyl, 1-butenyl and 2-butenyl.

The term “alkynyl” is used herein to mean a straight or branched chain radical of 2-10 carbon atoms, unless the chain length is limited thereto, wherein there is at least one triple bond between two of the carbon atoms in the chain. Typical alkynyl groups include ethynyl, 1-propynyl, 1-methyl-2-propynyl, 2-propynyl, 1-butynyl and 2-butynyl.

Useful alkoxy groups include oxygen substituted by one of the C₁₋₁₀ alkyl groups mentioned above, which may be optionally substituted. Alkoxy substituents include, without limitation, halo, morpholino, amino including alkylamino and dialkylamino, and carboxy including esters thereof.

Useful alkylthio groups include sulfur substituted by one of the C₁₋₁₀ alkyl groups mentioned above, which may be optionally substituted. Also included are the sulfoxides and sulfones of such alkylthio groups.

Useful amino and optionally substituted amino groups include —NH₂, —NHR₁₅ and —NR₁₅R₁₆, wherein R₁₅ and R₁₆ are C₁₋₁₀ alkyl or cycloalkyl groups, or R₁₅ and R₁₆ are combined with the N to form a ring structure, such as a piperidine, or R₁₅ and R₁₆ are combined with the N and other group to form a ring, such as a piperazine. The alkyl group may be optionally substituted.

Optional substituents on the alkyl, alkoxy, alkylthio, alkenyl, alkynyl, cycloalkyl, carbocyclic and heterocyclic groups include one or more halo, hydroxy, carboxyl, amino, nitro, cyano, C₁-C₆ acylamino, C₁-C₆ acyloxy, C₁-C₆ alkoxy, aryloxy, alkylthio, C₆-C₁₀ aryl, C₄-C₇ cycloalkyl, C₂-C₆ alkenyl, C₂-C₆ alkynyl, C₆-C₁₀ aryl(C₂-C₆)alkenyl, C₆-C₁₀ aryl(C₂-C₆)alkynyl, saturated and unsaturated heterocyclic or heteroaryl.

Optional substituents on the aryl, arylalkyl, arylalkenyl, arylalkynyl and heteroaryl and heteroarylalkyl groups include one or more halo, C₁-C₆ haloalkyl, C₆-C₁₀ aryl, C₄-C₇ cycloalkyl, C₁-C₆ alkyl, C₂-C₆ alkenyl, C₂-C₆ alkynyl, C₆-C₁₀ arylcarboxamido, C₆-C₁₀ aryl(C₁-C₆)alkyl, C₆-C₁₀ aryl(C₂-C₆)alkenyl, C₆-C₁₀ aryl(C₂-C₆)alkynyl, C₁-C₆ hydroxyalkyl, nitro, amino, ureido, cyano, C₁-C₆ acylamino, hydroxy, thiol, C₁-C₆ acyloxy, azido, C₁-C₆ alkoxy or carboxy.

The term “aryl” as employed herein by itself or as part of another group refers to monocyclic, bicyclic or tricyclic aromatic groups containing from 6 to 14 carbons in the ring portion.

Useful aryl groups include C₆₋₁₄ aryl, preferably C₆₋₁₀ aryl. Typical C₆₋₁₄ aryl groups include phenyl, naphthyl, phenanthrenyl, anthracenyl, indenyl, azulenyl, biphenyl, biphenylenyl and fluorenyl groups.

The term “carbocycle” as employed herein include cycloalkyl and partially saturated carbocyclic groups. Useful cycloalkyl groups are C₃₋₈ cycloalkyl. Typical cycloalkyl groups include cyclopropyl, cyclobutyl, cyclopentyl, cyclohexyl and cycloheptyl.

Useful saturated or partially saturated carbocyclic groups are cycloalkyl groups as described above, as well as cycloalkenyl groups, such as cyclopentenyl, cycloheptenyl and cyclooctenyl.

Useful halo or halogen groups include fluorine, chlorine, bromine and iodine.

The term “arylalkyl” is used herein to mean any of the above-mentioned C₁₋₁₀ alkyl groups substituted by any of the above-mentioned C₆₋₁₄ aryl groups. Preferably the arylalkyl group is benzyl, phenethyl or naphthylmethyl.

The term “arylalkenyl” is used herein to mean any of the above-mentioned C₂₋₁₀ alkenyl groups substituted by any of the above-mentioned C₆₋₁₄ aryl groups.

The term “arylalkynyl” is used herein to mean any of the above-mentioned C₂₋₁₀ alkynyl groups substituted by any of the above-mentioned C₆₋₁₄ aryl groups.

The term “aryloxy” is used herein to mean oxygen substituted by one of the above-mentioned C₆₋₁₄ aryl groups, which may be optionally substituted. Useful aryloxy groups include phenoxy and 4-methylphenoxy.

The term “arylalkoxy” is used herein to mean any of the above mentioned C₁₋₁₀ alkoxy groups substituted by any of the above-mentioned aryl groups, which may be optionally substituted. Useful arylalkoxy groups include benzyloxy and phenethyloxy.

Useful haloalkyl groups include C₁₋₁₀ alkyl groups substituted by one or more fluorine, chlorine, bromine or iodine atoms, e.g., fluoromethyl, difluoromethyl, trifluoromethyl, pentafluoroethyl, 1,1-difluoroethyl, chloromethyl, chlorofluoromethyl and trichloromethyl groups.

Useful acylamino (acylamido) groups are any C₁₋₆ acyl (alkanoyl) attached to an amino nitrogen, e.g., acetamido, chloroacetamido, propionamido, butanoylamido, pentanoylamido and hexanoylamido, as well as aryl-substituted C₁₋₆ acylamino groups, e.g., benzoylamido, and pentafluorobenzoylamido.

Useful acyloxy groups are any C₁₋₆ acyl (alkanoyl) attached to an oxy (—O—) group, e.g., formyloxy, acetoxy, propionoyloxy, butanoyloxy, pentanoyloxy and hexanoyloxy.

The term heterocycle is used herein to mean a saturated or partially saturated 3-7 membered monocyclic, or 7-10 membered bicyclic ring system, which consists of carbon atoms and from one to four heteroatoms independently selected from the group consisting of O, N, and S, wherein the nitrogen and sulfur heteroatoms can be optionally oxidized, the nitrogen can be optionally quaternized, and including any bicyclic group in which any of the above-defined heterocyclic rings is fused to a benzene ring, and wherein the heterocyclic ring can be substituted on carbon or on a nitrogen atom if the resulting compound is stable.

Useful saturated or partially saturated heterocyclic groups include tetrahydrofuranyl, pyranyl, piperidinyl, piperazinyl, pyrrolidinyl, imidazolidinyl, imidazolinyl, indolinyl, isoindolinyl, quinuclidinyl, morpholinyl, isochromanyl, chromanyl, pyrazolidinyl pyrazolinyl, tetronoyl and tetramoyl groups.

The term “heteroaryl” as employed herein refers to groups having 5 to 14 ring atoms; 6, 10 or 14 π electrons shared in a cyclic array; and containing carbon atoms and 1, 2 or 3 oxygen, nitrogen or sulfur heteroatoms.

Useful heteroaryl groups include thienyl (thiophenyl), benzo[b]thienyl, naphtho[2,3-b]thienyl, thianthrenyl, furyl (furanyl), pyranyl, isobenzofuranyl, chromenyl, xanthenyl, phenoxanthiinyl, pyrrolyl, including without limitation 2H-pyrrolyl, imidazolyl, pyrazolyl, pyridyl (pyridinyl), including without limitation 2-pyridyl, 3-pyridyl, and 4-pyridyl, pyrazinyl, pyrimidinyl, pyridazinyl, indolizinyl, isoindolyl, 3H-indolyl, indolyl, indazolyl, purinyl, 4H-quinolizinyl, isoquinolyl, quinolyl, phthalzinyl, naphthyridinyl, quinozalinyl, cinnolinyl, pteridinyl, carbazolyl, β-carbolinyl, phenanthridinyl, acrindinyl, perimidinyl, phenanthrolinyl, phenazinyl, isothiazolyl, phenothiazinyl, isoxazolyl, furazanyl, phenoxazinyl, 1,4-dihydroquinoxaline-2,3-dione, 7-aminoisocoumarin, pyrido[1,2-a]pyrimidin-4-one, pyrazolo[1,5-a]pyrimidinyl, including without limitation pyrazolo[1,5-a]pyrimidin-3-yl, 1,2-benzoisoxazol-3-yl, benzimidazolyl, 2-oxindolyl and 2-oxobenzimidazolyl. Where the heteroaryl group contains a nitrogen atom in a ring, such nitrogen atom may be in the form of an N-oxide, e.g., a pyridyl N-oxide, pyrazinyl N-oxide and pyrimidinyl N-oxide.

The term “heteroaryloxy” is used herein to mean oxygen substituted by one of the above-mentioned heteroaryl groups, which may be optionally substituted. Useful heteroaryloxy groups include pyridyloxy, pyrazinyloxy, pyrrolyloxy, pyrazolyloxy, imidazolyloxy and thiophenyloxy.

The term “heteroarylalkoxy” is used herein to mean any of the above-mentioned C₁₋₁₀ alkoxy groups substituted by any of the above-mentioned heteroaryl groups, which may be optionally substituted.

Some of the compounds of the present invention may exist as stereoisomers including optical isomers. The invention includes all stereoisomers and both the racemic mixtures of such stereoisomers as well as the individual enantiomers that may be separated according to methods that are well known to those of ordinary skill in the art.

Examples of pharmaceutically acceptable addition salts include inorganic and organic acid addition salts, such as hydrochloride, hydrobromide, phosphate, sulphate, citrate, lactate, tartrate, maleate, fumarate, mandelate and oxalate; and inorganic and organic base addition salts with bases, such as sodium hydroxy, Tris(hydroxymethyl)aminomethane (TRIS, tromethane) and N-methyl-glucamine.

Examples of prodrugs of the compounds of the invention include the simple esters of carboxylic acid containing compounds (e.g., those obtained by condensation with a C₁₋₄ alcohol according to methods known in the art); esters of hydroxy containing compounds (e.g., those obtained by condensation with a C₁₋₄ carboxylic acid, C₃₋₆ dioic acid or anhydride thereof, such as succinic and fumaric anhydrides according to methods known in the art); imines of amino containing compounds (e.g., those obtained by condensation with a C₁₋₄ aldehyde or ketone according to methods known in the art); carbamate of amino containing compounds, such as those described by Leu, et. al., (J. Med. Chem. 42:3623-3628 (1999)) and Greenwald, et. al., (J. Med. Chem. 42:3657-3667 (1999)); and acetals and ketals of alcohol containing compounds (e.g., those obtained by condensation with chloromethyl methyl ether or chloromethyl ethyl ether according to methods known in the art).

The compounds of this invention may be prepared using methods known to those skilled in the art, or the novel methods of this invention. Specifically, the compounds of this invention with Formulae I-V may be prepared as illustrated by the exemplary reaction in Scheme 1. Reaction of methyl glycolate with methyl acrylate in the presence of a base such as sodium hydride (NaH) produced methyl tetrahydro-4-oxofuran-3-carboxylate. Reaction of methyl tetrahydro-4-oxofuran-3-carboxylate with 2-methyl-2-thiopseudourea sulfate in the presence of a base such as potassium hydroxide (KOH) produced 5,7-dihydro-2-(methylthio)furo[3,4-d]pyrimidin-4-ol. Treatment of 5,7-dihydro-2-(methylthio)furo[3,4-d]pyrimidin-4-ol with phosphorous oxychloride produced 4-chloro-5,7-dihydro-2-(methylthio)furo[3,4-d]pyrimidine. Reaction of 4-chloro-5,7-dihydro-2-(methylthio)furo[3,4-d]pyrimidine with a substituted aniline such as 3,5-dimethoxyaniline in i-propanol (i-PrOH) produced 5,7-dihydro-N-(3,5-dimethoxyphenyl)-2-(methylthio)furo[3,4-d]pyrimidin-4-amine. Reaction of 5,7-dihydro-N-(3,5-dimethoxyphenyl)-2-(methylthio)furo[3,4-d]pyrimidin-4-amine with hydrogen (H₂) in the presence of Raney Ni produced 5,7-dihydro-N-(3,5-dimethoxyphenyl)-furo[3,4-d]pyrimidin-4-amine.

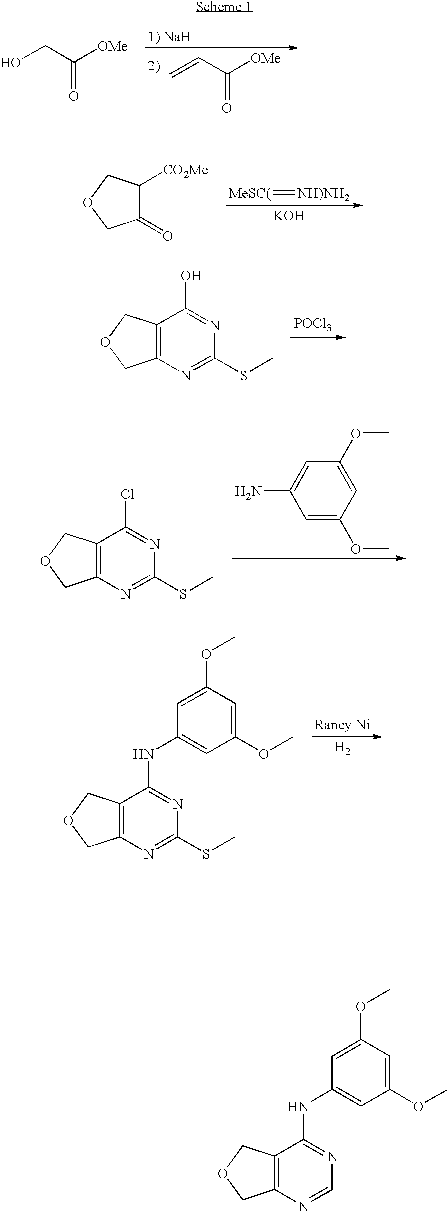

Compounds of this invention also may be prepared similarly as illustrated by the exemplary reaction in Scheme 2. Reaction of acetamidine hydrochloride with methyl tetrahydro-4-oxofuran-3-carboxylate produced 5,7-dihydro-2-methylfuro[3,4-d]pyrimidin-4-ol. Treatment of 5,7-dihydro-2-methylfuro[3,4-d]pyrimidin-4-ol with phosphorous oxychloride produced 4-chloro-5,7-dihydro-2-methylfuro[3,4-d]pyrimidine. Reaction of 4-chloro-5,7-dihydro-2-methylfuro[3,4-d]pyrimidine with a substituted aniline such as 3,5-dimethoxyaniline in i-propanol (i-PrOH) produced 5,7-dihydro-N-(3,5-dimethoxyphenyl)-2-methylfuro[3,4-d]pyrimidin-4-amine.

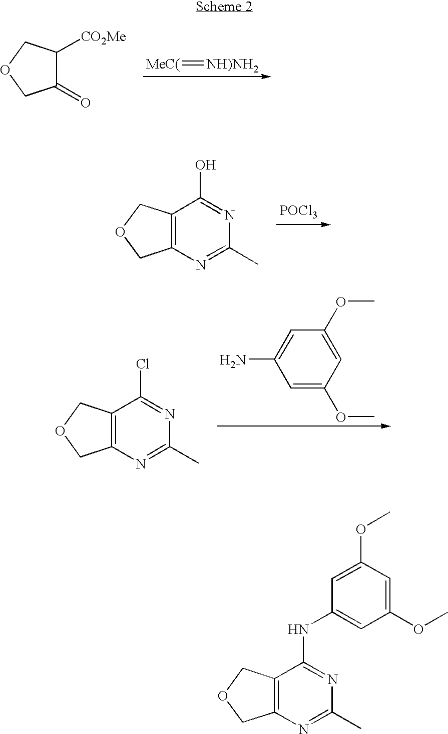

Similarly, other compounds of this invention may be prepared as illustrated by the exemplary reaction in Scheme 3. Reaction of 4-chloro-5,7-dihydro-2-(methylthio)furo[3,4-d]pyrimidine with a substituted aniline such as N-methyl-p-anisidine produced 5,7-dihydro-N-(4-methoxyphenyl)-N-methyl-2-(methylthio)furo[3,4-d]pyrimidin-4-amine.

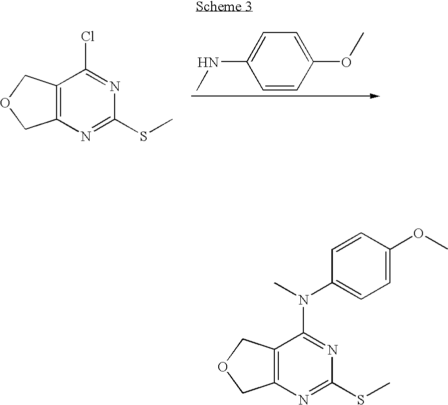

Compounds of this invention may be prepared as illustrated by the exemplary reaction in Scheme 4. Oxidation of 4-chloro-5,7-dihydro-2-(methylthio)furo[3,4-d]pyrimidine by m-chloroperbenzoic acid (mCPBA) produced 4-chloro-5,7-dihydro-2-(methylsulfonyl)furo[3,4-d]pyrimidine. Reaction of 4-chloro-5,7-dihydro-2-(methylsulfonyl)furo[3,4-d]pyrimidine with a substituted arylamine such as 3-methyl-1H-pyrazol-5-amine produced 5,7-dihydro-N-(3-methyl-1H-pyrazol-5-yl)-2-(methylsulfonyl)furo[3,4-d]pyrimidin-4-amine. Reaction of 5,7-dihydro-N-(3-methyl-1H-pyrazol-5-yl)-2-(methylsulfonyl)furo[3,4-d]pyrimidin-4-amine with N-(4-mercaptophenyl)cyclopropanecarboxamide produced N-(4-(4-(3-Methyl-1H-pyrazol-5-ylamino)-5,7-dihydrofuro[3,4-d]pyrimidin-2-ylthio)phenyl)cyclopropanecarboxamide.

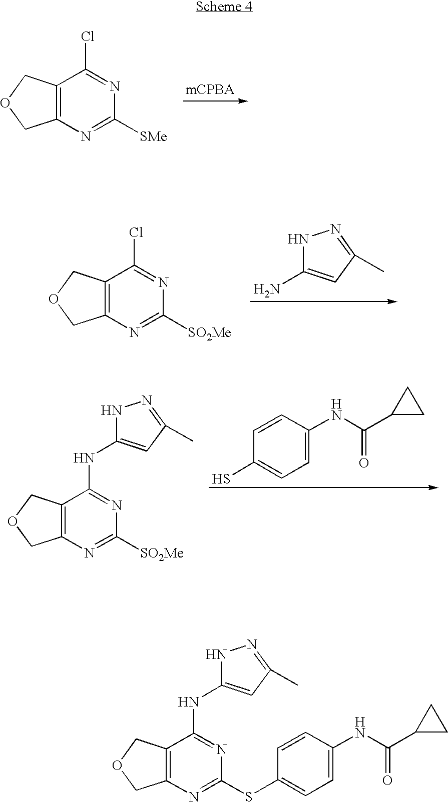

Compounds of this invention also may be prepared as illustrated by the exemplary reaction in Scheme 5. Reaction of 4-chloro-5,7-dihydro-2-(methylsulfonyl)furo[3,4-d]pyrimidine with N-(4-mercaptophenyl)cyclopropanecarboxamide produced N-(4-(5,7-dihydro-2-(methylsulfonyl)furo[3,4-d]pyrimidin-4-ylthio)phenyl)cyclopropanecarboxamide. Reaction of N-(4-(5,7-dihydro-2-(methylsulfonyl)furo[3,4-d]pyrimidin-4-ylthio)phenyl)cyclopropanecarboxamide with a substituted arylamine such as 3-methyl-1H-pyrazol-5-amine produced N-(4-(2-(3-Methyl-1H-pyrazol-5-ylamino)-5,7-dihydrofuro[3,4-d]pyrimidin-4-ylthio)phenyl)cyclopropanecarboxamide.

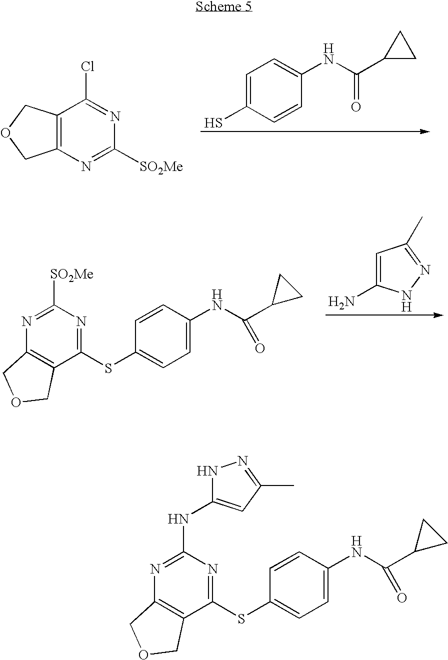

Compounds of this invention with Formulae I-V also may be prepared as illustrated by the exemplary reaction in Scheme 6. Reaction of methyl tetrahydro-4-oxothiophene-3-carboxylate with 2-methyl-2-thiopseudourea sulfate should produce 5,7-dihydro-2-(methylthio)thieno[3,4-d]pyrimidin-4-ol. Treatment of 5,7-dihydro-2-(methylthio)thieno[3,4-d]pyrimidin-4-ol with phosphorous oxychloride should produce 4-chloro-5,7-dihydro-2-(methylthio)thiono[3,4-d]pyrimidine. Reaction of 4-chloro-5,7-dihydro-2-(methylthio)thiono[3,4-d]pyrimidine with a substituted aniline such as 3,5-dimethoxyaniline should produce 5,7-dihydro-N-(3,5-dimethoxyphenyl)-2-(methylthio)thiono[3,4-d]pyrimidin-4-amine.

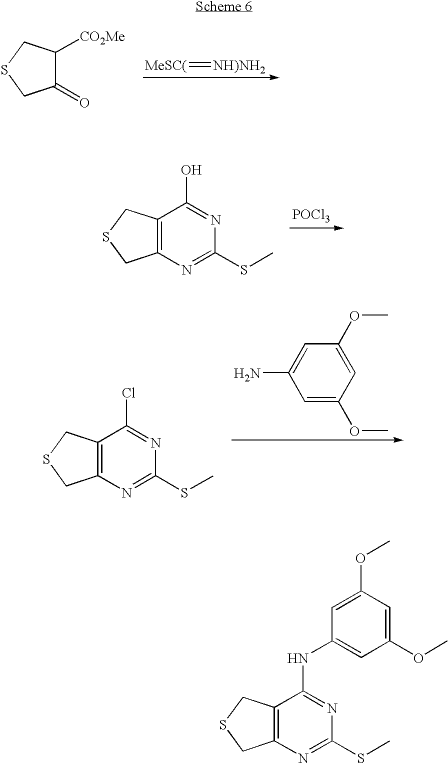

Compounds of this invention with Formulae I-V also may be prepared as illustrated by the exemplary reaction in Scheme 7. Reaction of (9H-fluoren-9-yl)methyl 2,4-dichloro-5H-pyrrolo[3,4-d]pyrimidine-6(7H)-carboxylate with a substituted arylamine such as 3-methyl-1H-pyrazol-5-amine produced (9H-fluoren-9-yl)methyl 4-(3-methyl-1H-pyrazol-5-ylamino)-2-chloro-5H-pyrrolo[3,4-d]pyrimidine-6(7H)-carboxylate. Reaction of (9H-fluoren-9-yl)methyl 4-(3-methyl-1H-pyrazol-5-ylamino)-2-chloro-5H-pyrrolo[3,4-d]pyrimidine-6(7H)-carboxylate with a substituted amine such as N-Me-piperazine produced 6,7-dihydro-N-(3-methyl-1H-pyrazol-5-yl)-2-(4-methylpiperazin-1-yl)-5H-pyrrolo[3,4-d]pyrimidin-4-amine.

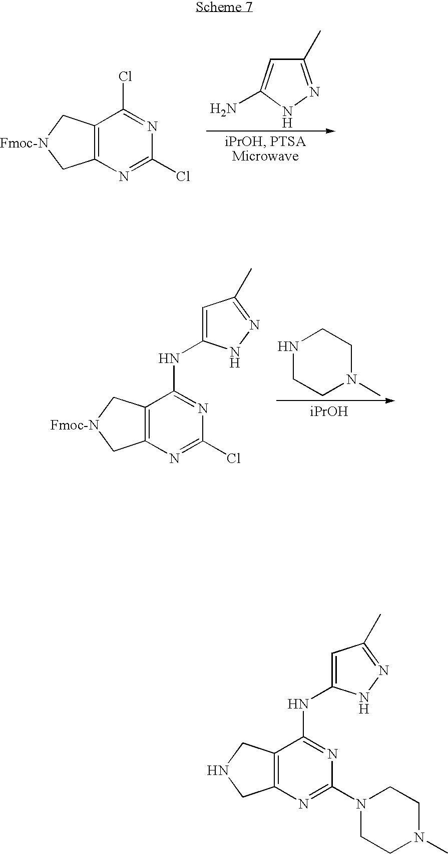

Similarly, compounds of this invention with Formulae I-V may be prepared as illustrated by the exemplary reaction in Scheme 8. Reaction of (9H-fluoren-9-yl)methyl 4-(3-methyl-1H-pyrazol-5-ylamino)-2-chloro-5H-pyrrolo[3,4-d]pyrimidine-6(7H)-carboxylate with N-(4-mercaptophenyl)cyclopropanecarboxamide should produce (9H-fluoren-9-yl)methyl 4-(3-methyl-1H-pyrazol-5-ylamino)-2-((4-(cyclopropanecarboxamido)phenyl)sulfanyl)-5H-pyrrolo[3,4-d]pyrimidine-6(7H)-carboxylate.

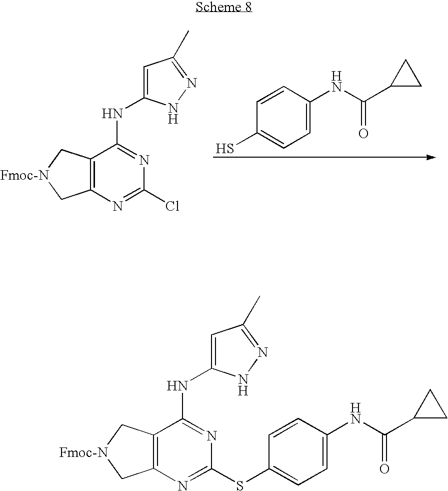

Similarly, compounds of this invention with Formulae I-V also may be prepared as shown in Scheme 9. Reaction of methyl 2-oxocyclopentanecarboxylate with 2-methyl-2-thiopseudourea sulfate in the presence of a base such as potassium hydroxide (KOH) produced 6,7-dihydro-2-(methylthio)-5H-cyclopenta[d]pyrimidin-4-ol, which was converted into 4-chloro-6,7-dihydro-2-(methylthio)-5H-cyclopenta[d]pyrimidine via treatment with phosphorous oxychloride. Reaction of 4-chloro-6,7-dihydro-2-(methylthio)-5H-cyclopenta[d]pyrimidine with a substituted aniline such as 3,5-dimethoxyaniline in i-propanol (i-PrOH) produced 6,7-dihydro-N-(2,5-dimethoxyphenyl)-2-(methylthio)-5H-cyclopenta[d]pyrimidin-4-amine. Reaction of 6,7-dihydro-N-(2,5-dimethoxyphenyl)-2-(methylthio)-5H-cyclopenta[d]pyrimidin-4-amine with hydrogen (H₂) in the presence of Raney Ni produced 6,7-dihydro-N-(2,5-dimethoxyphenyl)-5H-cyclopenta[d]pyrimidin-4-amine.

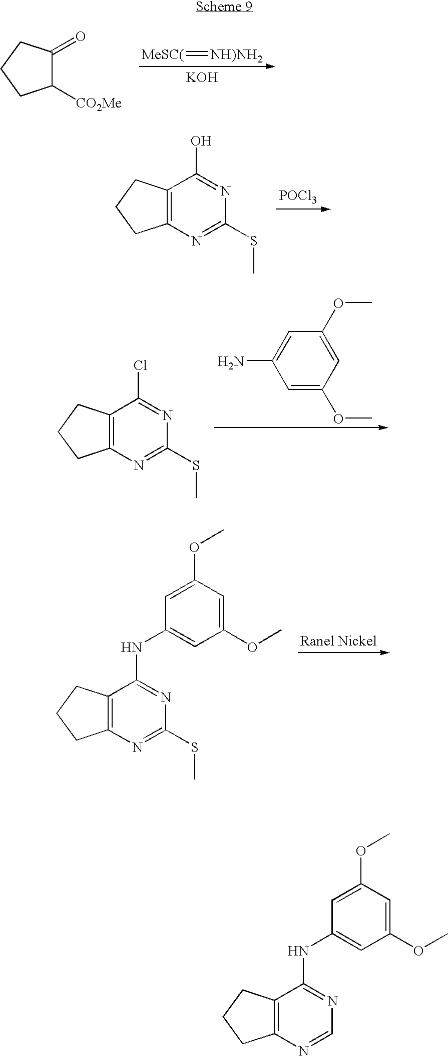

Similarly, compounds of this invention with Formulae I-V also may be prepared as shown in Scheme 10. Reaction of 6,7-dihydro-N-(2,5-dimethoxyphenyl)-2-(methylthio)-5H-cyclopenta[d]pyrimidin-4-amine with m-chloroperbenzoic acid (m-CPBA) produced 6,7-dihydro-N-(3,5-dimethoxyphenyl)-2-(methylsulfinyl)-5H-cyclopenta[d]pyrimidin-4-amine.

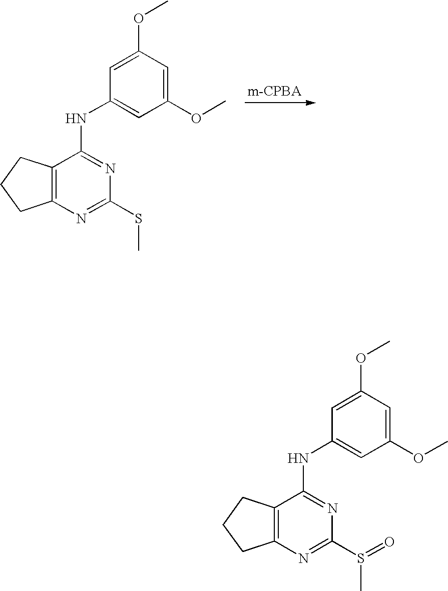

An important aspect of the present invention is the discovery that compounds having Formulae I-V are activators of caspases and inducers of apoptosis. Therefore, these compounds are useful in a variety of clinical conditions in which there is uncontrolled cell growth and spread of abnormal cells, such as in the case of cancer.

Another important aspect of the present invention is the discovery that compounds having Formulae I-V are potent and highly efficacious activators of caspases and inducers of apoptosis in drug resistant cancer cells, such as breast and prostate cancer cells, which enables these compounds to kill these drug resistant cancer cells. In comparison, most standard anti-cancer drugs are not effective in killing drug resistant cancer cells under the same conditions. Therefore, compounds of this invention are useful for the treatment of drug resistant cancer, such as breast cancer in animals.

The present invention includes a therapeutic method useful to modulate in vivo apoptosis or in vivo neoplastic disease, comprising administering to a subject in need of such treatment an effective amount of a compound, or a pharmaceutically acceptable salt or prodrug of the compound of Formulae I-V, which functions as a caspase cascade activator and inducer of apoptosis.

The present invention also includes a therapeutic method comprising administering to an animal an effective amount of a compound, or a pharmaceutically acceptable salt or prodrug of said compound of Formulae I-V, wherein said therapeutic method is useful to treat cancer, which is a group of diseases characterized by the uncontrolled growth and spread of abnormal cells. Such diseases include, but are not limited to, Hodgkin's disease, non-Hodgkin's lymphoma, acute lymphocytic leukemia, chronic lymphocytic leukemia, multiple myeloma, neuroblastoma, breast carcinoma, ovarian carcinoma, lung carcinoma, Wilms' tumor, cervical carcinoma, testicular carcinoma, soft-tissue sarcoma, primary macroglobulinemia, bladder carcinoma, chronic granulocytic leukemia, primary brain carcinoma, malignant melanoma, small-cell lung carcinoma, stomach carcinoma, colon carcinoma, malignant pancreatic insulinoma, malignant carcinoid carcinoma, choriocarcinoma, mycosis fungoides, head or neck carcinoma, osteogenic sarcoma, pancreatic carcinoma, acute granulocytic leukemia, hairy cell leukemia, neuroblastoma, rhabdomyosarcoma, Kaposi's sarcoma, genitourinary carcinoma, thyroid carcinoma, esophageal carcinoma, malignant hypercalcemia, cervical hyperplasia, renal cell carcinoma, endometrial carcinoma, polycythemia vera, essential thrombocytosis, adrenal cortex carcinoma, skin cancer, and prostatic carcinoma.

In practicing the therapeutic methods, effective amounts of compositions containing therapeutically effective concentrations of the compounds formulated for oral, intravenous, local and topical application, for the treatment of neoplastic diseases and other diseases in which caspase cascade mediated physiological responses are implicated, are administered to an individual exhibiting the symptoms of one or more of these disorders. The amounts are effective to ameliorate or eliminate one or more symptoms of the disorders. An effective amount of a compound for treating a particular disease is an amount that is sufficient to ameliorate, or in some manner reduce, the symptoms associated with the disease. Such amount may be administered as a single dosage or may be administered according to a regimen, whereby it is effective. The amount may cure the disease but, typically, is administered in order to ameliorate the symptoms of the disease. Typically, repeated administration is required to achieve the desired amelioration of symptoms.

In another embodiment, a pharmaceutical composition comprising a compound, or a pharmaceutically acceptable salt of said compound of Formulae I-V, which functions as a caspase cascade activator and inducer of apoptosis in combination with a pharmaceutically acceptable vehicle is provided.

Another embodiment of the present invention is directed to a composition effective to inhibit neoplasia comprising a compound, or a pharmaceutically acceptable salt or prodrug of said compound of Formulae I-V, which functions as a caspase cascade activator and inducer of apoptosis, in combination with at least one known cancer chemotherapeutic agent, or a pharmaceutically acceptable salt of said agent. Examples of known cancer chemotherapeutic agents which may be used for combination therapy include, but not are limited to alkylating agents, such as busulfan, cis-platin, mitomycin C, and carboplatin; antimitotic agents, such as colchicine, vinblastine, paclitaxel, and docetaxel; topo I inhibitors, such as camptothecin and topotecan; topo II inhibitors, such as doxorubicin and etoposide; RNA/DNA antimetabolites, such as 5-azacytidine, 5-fluorouracil and methotrexate; DNA antimetabolites, such as 5-fluoro-2′-deoxy-uridine, ara-C, hydroxyurea and thioguanine; antibodies, such as campath, Herceptin® or Rituxan®. Other known cancer chemotherapeutic agents which may be used for combination therapy include melphalan, chlorambucil, cyclophosamide, ifosfamide, vincristine, mitoguazone, epirubicin, aclarubicin, bleomycin, mitoxantrone, elliptinium, fludarabine, octreotide, retinoic acid, tamoxifen, Gleevec® and alanosine.

In practicing the methods of the present invention, the compound of the invention may be administered together with at least one known chemotherapeutic agent as part of a unitary pharmaceutical composition. Alternatively, the compound of the invention may be administered apart from at least one known cancer chemotherapeutic agent. In one embodiment, the compound of the invention and at least one known cancer chemotherapeutic agent are administered substantially simultaneously, i.e. the compounds are administered at the same time or one after the other, so long as the compounds reach therapeutic levels in the blood at the same time. On another embodiment, the compound of the invention and at least one known cancer chemotherapeutic agent are administered according to their individual dose schedule, so long as the compounds reach therapeutic levels in the blood.

It has been reported that alpha-1-adrenoceptor antagonists, such as doxazosin, terazosin, and tamsulosin can inhibit the growth of prostate cancer cell via induction of apoptosis (Kyprianou, N., et al., Cancer Res 60:4550-4555, (2000)). Therefore, another embodiment of the present invention is directed to a composition effective to inhibit neoplasia comprising a compound, or a pharmaceutically acceptable salt or prodrug of a compound described herein, which functions as a caspase cascade activator and inducer of apoptosis, in combination with at least one known alpha-1-adrenoceptor antagonists, or a pharmaceutically acceptable salt of said agent. Examples of known alpha-1-adrenoceptor antagonists, which can be used for combination therapy include, but are not limited to, doxazosin, terazosin, and tamsulosin.

It has been reported that sigma-2 receptors are expressed in high densities in a variety of tumor cell types (Vilner, B. J., et al., Cancer Res. 55: 408-413 (1995)) and that sigma-2 receptor agonists, such as CB-64D, CB-184 and haloperidol activate a novel apoptotic pathway and potentiate antineoplastic drugs in breast tumor cell lines. (Kyprianou, N., et al., Cancer Res. 62:313-322 (2002)). Therefore, another embodiment of the present invention is directed to a composition effective to inhibit neoplasia comprising a compound, or a pharmaceutically acceptable salt or prodrug of a compound described herein, which functions as a caspase cascade activator and inducer of apoptosis, in combination with at least one known sigma-2 receptor agonist, or a pharmaceutically acceptable salt of said agonist. Examples of known sigma-2 receptor agonists that can be used for combination therapy include, but are not limited to, CB-64D, CB-184 and haloperidol.

It has been reported that combination therapy with lovastatin, a HMG-CoA reductase inhibitor, and butyrate, an inducer of apoptosis in the Lewis lung carcinoma model in mice, showed potentiating antitumor effects (Giermasz, A., et al., Int. J. Cancer 97:746-750 (2002)). Therefore, another embodiment of the present invention is directed to a composition effective to inhibit neoplasia comprising a compound, or a pharmaceutically acceptable salt or prodrug of a compound described herein, which functions as a caspase cascade activator and inducer of apoptosis, in combination with at least one known HMG-CoA reductase inhibitor, or a pharmaceutically acceptable salt of said agent. Examples of known HMG-CoA reductase inhibitors, which can be used for combination therapy include, but are not limited to, lovastatin, simvastatin, pravastatin, fluvastatin, atorvastatin, and cerivastatin.

It has been reported that HIV protease inhibitors, such as indinavir or saquinavir, have potent anti-angiogenic activities and promote regression of Kaposi sarcoma (Sgadari, C., et al., Nat. Med. 8:225-232 (2002)). Therefore, another embodiment of the present invention is directed to a composition effective to inhibit neoplasia comprising a compound, or a pharmaceutically acceptable salt or prodrug of a compound described herein, which functions as a caspase cascade activator and inducer of apoptosis, in combination with at least one known HIV protease inhibitor, or a pharmaceutically acceptable salt of said agent. Examples of known HIV protease inhibitors, which can be used for combination therapy include, but are not limited to, amprenavir, abacavir, CGP-73547, CGP-61755, DMP-450, indinavir, nelfinavir, tipranavir, ritonavir, saquinavir, ABT-378, AG 1776, and BMS-232,632.

It has been reported that synthetic retinoids, such as fenretinide (N-(4-hydroxyphenyl)retinamide, 4HPR), have good activity in combination with other chemotherapeutic agents, such as cisplatin, etoposide or paclitaxel in small-cell lung cancer cell lines (Kalemkerian, G. P., et al., Cancer Chemother. Pharmacol. 43:145-150 (1999)). 4HPR also was reported to have good activity in combination with gamma-radiation on bladder cancer cell lines (Zou, C., et al., Int. J. Oncol. 13: 1037-1041 (1998)). Therefore, another embodiment of the present invention is directed to a composition effective to inhibit neoplasia comprising a compound, or a pharmaceutically acceptable salt or prodrug of a compound described herein, which functions as a caspase cascade activator and inducer of apoptosis, in combination with at least one known retinoid and synthetic retinoid, or a pharmaceutically acceptable salt of said agent. Examples of known retinoids and synthetic retinoids, which can be used for combination therapy include, but are not limited to, bexarotene, tretinoin, 13-cis-retinoic acid, 9-cis-retinoic acid, α-difluoromethylornithine, ILX23-7553, fenretinide, and N-4-carboxyphenyl retinamide.

It has been reported that proteasome inhibitors, such as lactacystin, exert anti-tumor activity in vivo and in tumor cells in vitro, including those resistant to conventional chemotherapeutic agents. By inhibiting NF-kappaB transcriptional activity, proteasome inhibitors may also prevent angiogenesis and metastasis in vivo and further increase the sensitivity of cancer cells to apoptosis (Almond, J. B., et al., Leukemia 16:433-443 (2002)). Therefore, another embodiment of the present invention is directed to a composition effective to inhibit neoplasia comprising a compound, or a pharmaceutically acceptable salt or prodrug of a compound described herein, which functions as a caspase cascade activator and inducer of apoptosis, in combination with at least one known proteasome inhibitor, or a pharmaceutically acceptable salt of said agent. Examples of known proteasome inhibitors, which can be used for combination therapy include, but are not limited to, lactacystin, MG-132, and PS-341.

It has been reported that tyrosine kinase inhibitors, such as ST1571 (Imatinib mesilate, Gleevec®), have potent synergetic effect in combination with other anti-leukemic agents, such as etoposide (Liu, W. M., et al. Br. J. Cancer 86:1472-1478 (2002)). Therefore, another embodiment of the present invention is directed to a composition effective to inhibit neoplasia comprising a compound, or a pharmaceutically acceptable salt or prodrug of a compound described herein, which functions as a caspase cascade activator and inducer of apoptosis, in combination with at least one known tyrosine kinase inhibitor, or a pharmaceutically acceptable salt of said agent. Examples of known tyrosine kinase inhibitors, which can be used for combination therapy include, but are not limited to, Gleevec®, ZD1839 (Iressa), SH268, genistein, CEP2563, SU6668, SU11248, and EMD121974.

It has been reported that prenyl-protein transferase inhibitors, such as farnesyl protein transferase inhibitor R115777, possess preclinical antitumor activity against human breast cancer (Kelland, L. R., et. al., Clin. Cancer Res. 7:3544-3550 (2001)). Synergy of the protein farnesyltransferase inhibitor SCH66336 and cisplatin in human cancer cell lines also has been reported (Adjei, A. A., et al., Clin. Cancer. Res. 7:1438-1445 (2001)). Therefore, another embodiment of the present invention is directed to a composition effective to inhibit neoplasia comprising a compound, or a pharmaceutically acceptable salt or prodrug of a compound described herein, which functions as a caspase cascade activator and inducer of apoptosis, in combination with at least one known prenyl-protein transferase inhibitor, including farnesyl protein transferase inhibitor, inhibitors of geranylgeranyl-protein transferase type I (GGPTase-I) and geranylgeranyl-protein transferase type-II, or a pharmaceutically acceptable salt of said agent. Examples of known prenyl-protein transferase inhibitors, which can be used for combination therapy include, but are not limited to, R115777, SCH66336, L-778,123, BAL9611 and TAN-1813.

It has been reported that cyclin-dependent kinase (CDK) inhibitors, such as flavopiridol, have potent synergetic effect in combination with other anticancer agents, such as CPT-11, a DNA topoisomerase I inhibitor in human colon cancer cells (Motwani, M., et al., Clin. Cancer Res. 7:4209-4219, (2001)). Therefore, another embodiment of the present invention is directed to a composition effective to inhibit neoplasia comprising a compound, or a pharmaceutically acceptable salt or prodrug of a compound described herein, which functions as a caspase cascade activator and inducer of apoptosis, in combination with at least one known cyclin-dependent kinase inhibitor, or a pharmaceutically acceptable salt of said agent. Examples of known cyclin-dependent kinase inhibitor, which can be used for combination therapy include, but are not limited to, flavopiridol, UCN-01, roscovitine, and olomoucine.

It has been reported that in preclinical studies COX-2 inhibitors were found to block angiogenesis, suppress solid tumor metastases, and slow the growth of implanted gastrointestinal cancer cells (Blanke, C. D., Oncology (Huntingt) 16 (No. 4 Suppl. 3):17-21 (2002)). Therefore, another embodiment of the present invention is directed to a composition effective to inhibit neoplasia comprising a compound, or a pharmaceutically acceptable salt or prodrug of a compound described herein, which functions as a caspase cascade activator and inducer of apoptosis, in combination with at least one known COX-2 inhibitor, or a pharmaceutically acceptable salt of said inhibitor. Examples of known COX-2 inhibitors that can be used for combination therapy include, but are not limited to, celecoxib, valecoxib, and rofecoxib.

Another embodiment of the present invention is directed to a composition effective to inhibit neoplasia comprising a bioconjugate of a compound described herein, which functions as a caspase cascade activator and inducer of apoptosis, in bioconjugation with at least one known therapeutically useful antibody, such as Herceptin® or Rituxan®, growth factors, such as DGF, NGF; cytokines, such as IL-2, IL-4, or any molecule that binds to the cell surface. The antibodies and other molecules will deliver a compound described herein to its targets and make it an effective anticancer agent. The bioconjugates could also enhance the anticancer effect of therapeutically useful antibodies, such as Herceptin® or Rituxan®.

Similarly, another embodiment of the present invention is directed to a composition effective to inhibit neoplasia comprising a compound, or a pharmaceutically acceptable salt or prodrug of a compound described herein, which functions as a caspase cascade activator and inducer of apoptosis, in combination with radiation therapy. In this embodiment, the compound of the invention may be administered at the same time as the radiation therapy is administered or at a different time.

Yet another embodiment of the present invention is directed to a composition effective for post-surgical treatment of cancer, comprising a compound, or a pharmaceutically acceptable salt or prodrug of a compound described herein, which functions as a caspase cascade activator and inducer of apoptosis. The invention also relates to a method of treating cancer by surgically removing the cancer and then treating the animal with one of the pharmaceutical compositions described herein.

A wide range of immune mechanisms operates rapidly following exposure to an infectious agent. Depending on the type of infection, rapid clonal expansion of the T and B lymphocytes occurs to combat the infection. The elimination of the effector cells following an infection is one of the major mechanisms for maintaining immune homeostasis. Apoptosis has been shown to regulate the elimination of effector cells. Autoimmune diseases have lately been determined to occur as a consequence of deregulated cell death. In certain autoimmune diseases, the immune system directs its powerful cytotoxic effector mechanisms against specialized cells, such as oligodendrocytes in multiple sclerosis, the beta cells of the pancreas in diabetes mellitus, and thyrocytes in Hashimoto's thyroiditis (Ohsako, S. & Elkon, K. B., Cell Death Differ. 6:13-21 (1999)). Mutations of the gene encoding the lymphocyte apoptosis receptor Fas/APO-1/CD95 are reported to be associated with defective lymphocyte apoptosis and autoimmune lymphoproliferative syndrome (ALPS), which is characterized by chronic, histologically benign splenomegaly, generalized lymphadenopathy, hypergammaglobulinemia, and autoantibody formation. (Infante, A. J., et al., J. Pediatr. 133:629-633 (1998) and Vaishnaw, A. K., et al., J. Clin. Invest. 103:355-363 (1999)). It was reported that overexpression of Bc1-2, which is a member of the bcl-2 gene family of programmed cell death regulators with anti-apoptotic activity, in developing B cells of transgenic mice, in the presence of T cell dependent costimulatory signals, results in the generation of a modified B cell repertoire and in the production of pathogenic autoantibodies (Lopez-Hoyos, M., et al., Int. J. Mol. Med. 1:475-483 (1998)). It is therefore evident that many types of autoimmune disease are caused by defects of the apoptotic process. One treatment strategy for such diseases is to turn on apoptosis in the lymphocytes that are causing the autoimmune disease (O'Reilly, L. A. & Strasser, A., Inflamm. Res. 48:5-21 (1999)).

Fas-Fas ligand (FasL) interaction is known to be required for the maintenance of immune homeostasis. Experimental autoimmune thyroiditis (EAT), characterized by autoreactive T and B cell responses and a marked lymphocytic infiltration of the thyroid, is a good model to study the therapeutic effects of FasL. Batteux, F., et al., (J. Immunol. 162:603-608 (1999)) reported that by direct injection of DNA expression vectors encoding FasL into the inflamed thyroid, the development of lymphocytic infiltration of the thyroid was inhibited and induction of infiltrating T cells death was observed. These results show that FasL expression on thyrocytes may have a curative effect on ongoing EAT by inducing death of pathogenic autoreactive infiltrating T lymphocytes.

Bisindolylmaleimide VIII is known to potentiate Fas-mediated apoptosis in human astrocytoma 1321N1 cells and in Molt-4T cells; both of which were resistant to apoptosis induced by anti-Fas antibody in the absence of bisindolylmaleimide VIII. Potentiation of Fas-mediated apoptosis by bisindolylmaleimide VIII was reported to be selective for activated, rather than non-activated, T cells, and was Fas-dependent. Zhou T., et al., (Nat. Med. 5:42-48 (1999)) reported that administration of bisindolylmaleimide VIII to rats during autoantigen stimulation prevented the development of symptoms of T cell-mediated autoimmune diseases in two models, the Lewis rat model of experimental allergic encephalitis and the Lewis adjuvant arthritis model. Therefore, the application of a Fas-dependent apoptosis enhancer, such as bisindolylmaleimide VIII, may be therapeutically useful for the more effective elimination of detrimental cells and inhibition of T cell-mediated autoimmune diseases. Therefore, an effective amount of a compound, or a pharmaceutically acceptable salt or prodrug of the compound of Formulae I-V, which functions as a caspase cascade activator and inducer of apoptosis, is an effective treatment for autoimmune diseases.

Psoriasis is a chronic skin disease that is characterized by scaly red patches. Psoralen plus ultraviolet A (PUVA) is a widely used and effective treatment for psoriasis vulgaris. Coven, et al., Photodermatol. Photoimmunol. Photomed. 15:22-27 (1999), reported that lymphocytes treated with psoralen 8-MOP or TMP and UVA, displayed DNA degradation patterns typical of apoptotic cell death. Ozawa, et al., J. Exp. Med. 189:711-718 (1999) reported that induction of T cell apoptosis could be the main mechanism by which 312-nm UVB resolves psoriasis skin lesions. Low doses of methotrexate may be used to treat psoriasis to restore a clinically normal skin. Heenen, et al., Arch. Dermatol. Res. 290:240-245 (1998), reported that low doses of methotrexate may induce apoptosis and that this mode of action could explain the reduction in epidermal hyperplasia during treatment of psoriasis with methotrexate. Therefore, an effective amount of a compound, or a pharmaceutically acceptable salt or prodrug of the compound of Formulae I-V, which functions as a caspase cascade activator and inducer of apoptosis, is an effective treatment for hyperproliferative skin diseases, such as psoriasis.

Synovial cell hyperplasia is a characteristic of patients with rheumatoid arthritis (RA). It is believed that excessive proliferation of RA synovial cells, as well as defects in synovial cell death, may be responsible for synovial cell hyperplasia. Wakisaka, et al., Clin. Exp. Immunol. 114:119-128 (1998), found that although RA synovial cells could die via apoptosis through a Fas/FasL pathway, apoptosis of synovial cells was inhibited by proinflammatory cytokines present within the synovium. Wakisaka, et al. also suggested that inhibition of apoptosis by the proinflammatory cytokines may contribute to the outgrowth of synovial cells, and lead to pannus formation and the destruction of joints in patients with RA. Therefore, an effective amount of a compound, or a pharmaceutically acceptable salt or prodrug of the compound of Formulae I-V, which functions as a caspase cascade activator and inducer of apoptosis, is an effective treatment for rheumatoid arthritis.

There has been an accumulation of convincing evidence that apoptosis plays a major role in promoting resolution of the acute inflammatory response. Neutrophils are constitutively programmed to undergo apoptosis, thus limiting their pro-inflammatory potential and leading to rapid, specific, and non-phlogistic recognition by macrophages and semi-professional phagocytes (Savill, J., J. Leukoc. Biol. 61:375-380 (1997)). Boirivant, et al., Gastroenterology 116:557-565 (1999), reported that lamina propria T cells, isolated from areas of inflammation in Crohn's disease, ulcerative colitis, and other inflammatory states, manifest decreased CD2 pathway-induced apoptosis. In addition, studies of cells from inflamed Crohn's disease tissue indicate that elevated Bcl-2 levels accompany this defect. Therefore, an effective amount of a compound, or a pharmaceutically acceptable salt or prodrug of the compound of Formulae I-V, which functions as a caspase cascade activator and inducer of apoptosis, is an effective treatment for inflammation.

Caspase cascade activators and inducers of apoptosis may also be a desirable therapy in the elimination of pathogens, such as HIV, Hepatitis C and other viral pathogens. The long lasting quiecence, followed by disease progression, may be explained by an anti-apoptotic mechanism of these pathogens leading to persistent cellular reservoirs of the virions. It has been reported that HIV-1 infected T leukemia cells or peripheral blood mononuclear cells (PBMCs) underwent enhanced viral replication in the presence of the caspase inhibitor Z-VAD-fmk. Furthermore, Z-VAD-fmk also stimulated endogenous virus production in activated PBMCs derived from HIV-1-infected asymptomatic individuals (Chinnaiyan, A., et al., Nat. Med. 3:333 (1997)). Therefore, apoptosis serves as a beneficial host mechanism to limit the spread of HIV and new therapeutics using caspase/apoptosis activators are useful to clear viral reservoirs from the infected individuals. Similarly, HCV infection also triggers anti-apoptotic mechanisms to evade the host's immune surveillance leading to viral persistence and hepatocarcinogenesis (Tai, D. I., et al. Hepatology 3:656-64 (2000)). Therefore, apoptosis inducers are useful as therapeutics for HIV and other infectious disease.

Stent implantation has become the new standard angioplasty procedure. However, in-stent restenosis remains the major limitation of coronary stenting. New approaches have been developed to target pharmacological modulation of local vascular biology by local administration of drugs. This allows for drug applications at the precise site and time of vessel injury. Numerous pharmacological agents with antiproliferative properties are currently under clinical investigation, including actinomycin D, rapamycin or paclitaxel coated stents (Regar E., et al., Br. Med. Bull. 59:227-248 (2001)). Therefore, apoptosis inducers, which are antiproliferative, are useful as therapeutics for the prevention or reduction of in-stent restenosis.

Pharmaceutical compositions within the scope of this invention include all compositions wherein the compounds of the present invention are contained in an amount that is effective to achieve its intended purpose. While individual needs vary, determination of optimal ranges of effective amounts of each component is within the skill of the art. Typically, the compounds may be administered to animals, e.g., mammals, orally at a dose of 0.0025 to 50 mg/kg of body weight, per day, or an equivalent amount of the pharmaceutically acceptable salt thereof, to a mammal being treated. Preferably, approximately 0.01 to approximately 10 mg/kg of body weight is orally administered. For intramuscular injection, the dose is generally approximately one-half of the oral dose. For example, a suitable intramuscular dose would be approximately 0.0025 to approximately 25 mg/kg of body weight, and most preferably, from approximately 0.01 to approximately 5 mg/kg of body weight. If a known cancer chemotherapeutic agent is also administered, it is administered in an amount that is effective to achieve its intended purpose. The amounts of such known cancer chemotherapeutic agents effective for cancer are well known to those skilled in the art.

The unit oral dose may comprise from approximately 0.01 to approximately 50 mg, preferably approximately 0.1 to approximately 10 mg of the compound of the invention. The unit dose may be administered one or more times daily, as one or more tablets, each containing from approximately 0.1 to approximately 10 mg, conveniently approximately 0.25 to 50 mg of the compound or its solvates.

In a topical formulation, the compound may be present at a concentration of approximately 0.01 to 100 mg per gram of carrier.

In addition to administering the compound as a raw chemical, the compounds of the invention may be administered as part of a pharmaceutical preparation containing suitable pharmaceutically acceptable carriers comprising excipients and auxiliaries, which facilitate processing of the compounds into preparations that may be used pharmaceutically. Preferably, the preparations, particularly those preparations which may be administered orally and that may be used for the preferred type of administration, such as tablets, dragees, and capsules, and also preparations that may be administered rectally, such as suppositories, as well as suitable solutions for administration by injection or orally, contain from approximately 0.01 to 99 percent, preferably from approximately 0.25 to 75 percent of active compound(s), together with the excipient.

Also included within the scope of the present invention are the non-toxic pharmaceutically acceptable salts of the compounds of the present invention. Acid addition salts are formed by mixing a solution of the compounds of the present invention with a solution of a pharmaceutically acceptable non-toxic acid, such as hydrochloric acid, fumaric acid, maleic acid, succinic acid, acetic acid, citric acid, tartaric acid, carbonic acid, phosphoric acid, oxalic acid, and the like. Basic salts are formed by mixing a solution of the compounds of the present invention with a solution of a pharmaceutically acceptable non-toxic base, such as sodium hydroxide, potassium hydroxide, choline hydroxide, sodium carbonate, Tris, N-methyl-glucamine, and the like.

The pharmaceutical compositions of the invention may be administered to any animal, which may experience the beneficial effects of the compounds of the invention. Foremost among such animals are mammals, e.g., humans and veterinary animals, although the invention is not intended to be so limited.

The pharmaceutical compositions of the present invention may be administered by any means that achieve their intended purpose. For example, administration may be by parenteral, subcutaneous, intravenous, intramuscular, intraperitoneal, transdermal, buccal, intrathecal, intracranial, intranasal, or topical routes. Alternatively, or concurrently, administration may be by the oral route. The dosage administered will be dependent upon the age, health, and weight of the recipient, kind of concurrent treatment, if any, frequency of treatment, and the nature of the effect desired.

The pharmaceutical preparations of the present invention are manufactured in a manner, which is itself known, e.g., by means of conventional mixing, granulating, dragee-making, dissolving, or lyophilizing processes. Thus, pharmaceutical preparations for oral use may be obtained by combining the active compounds with solid excipients, optionally grinding the resulting mixture and processing the mixture of granules, after adding suitable auxiliaries, if desired or necessary, to obtain tablets or dragee cores.

Suitable excipients are, in particular: fillers, such as saccharides, e.g. lactose or sucrose, mannitol or sorbitol; cellulose preparations and/or calcium phosphates, e.g. tricalcium phosphate or calcium hydrogen phosphate; as well as binders, such as starch paste, using, e.g., maize starch, wheat starch, rice starch, potato starch, gelatin, tragacanth, methyl cellulose, hydroxypropylmethylcellulose, sodium carboxymethylcellulose, and/or polyvinyl pyrrolidone. If desired, disintegrating agents may be added, such as the above-mentioned starches and also carboxymethyl-starch, cross-linked polyvinyl pyrrolidone, agar, or alginic acid or a salt thereof, such as sodium alginate. Auxiliaries are, above all, flow-regulating agents and lubricants, e.g., silica, talc, stearic acid, or salts thereof, such as magnesium stearate or calcium stearate, and/or polyethylene glycol. Dragee cores are provided with suitable coatings that, if desired, are resistant to gastric juices. For this purpose, concentrated saccharide solutions may be used, which may optionally contain gum arabic, talc, polyvinyl pyrrolidone, polyethylene glycol and/or titanium dioxide, lacquer solutions and suitable organic solvents or solvent mixtures. In order to produce coatings resistant to gastric juices, solutions of suitable cellulose preparations, such as acetylcellulose phthalate or hydroxypropylmethyl-cellulose phthalate, are used. Dye stuffs or pigments may be added to the tablets or dragee coatings, e.g., for identification or in order to characterize combinations of active compound doses.

Other pharmaceutical preparations, which may be used orally, include push-fit capsules made of gelatin, as well as soft, sealed capsules made of gelatin and a plasticizer, such as glycerol or sorbitol. The push-fit capsules may contain the active compounds in the form of: granules, which may be mixed with fillers, such as lactose; binders, such as starches; and/or lubricants, such as talc or magnesium stearate and, optionally, stabilizers. In soft capsules, the active compounds are preferably dissolved or suspended in suitable liquids, such as fatty oils, or liquid paraffin. In addition, stabilizers may be added.

Possible pharmaceutical preparations, which may be used rectally include, e.g., suppositories, which consist of a combination of one or more of the active compounds with a suppository base. Suitable suppository bases are, e.g., natural or synthetic triglycerides, or paraffin hydrocarbons. In addition, it is also possible to use gelatin rectal capsules, which consist of a combination of the active compounds with a base. Possible base materials include, e.g., liquid triglycerides, polyethylene glycols, or paraffin hydrocarbons.

Suitable formulations for parenteral administration include aqueous solutions of the active compounds in water-soluble form, e.g., water-soluble salts and alkaline solutions. In addition, suspensions of the active compounds as appropriate oily injection suspensions may be administered. Suitable lipophilic solvents or vehicles include fatty oils, e.g., sesame oil, or synthetic fatty acid esters, e.g., ethyl oleate or triglycerides or polyethylene glycol-400 (the compounds are soluble in PEG-400), or cremophor, or cyclodextrins. Aqueous injection suspensions may contain substances which increase the viscosity of the suspension include, e.g., sodium carboxymethyl cellulose, sorbitol, and/or dextran. Optionally, the suspension may also contain stabilizers.

In accordance with one aspect of the present invention, compounds of the invention are employed in topical and parenteral formulations and are used for the treatment of skin cancer.

The topical compositions of this invention are formulated preferably as oils, creams, lotions, ointments, and the like by choice of appropriate carriers. Suitable carriers include vegetable or mineral oils, white petrolatum (white soft paraffin), branched chain fats or oils, animal fats and high molecular weight alcohol (greater than C₁₂). The preferred carriers are those in which the active ingredient is soluble. Emulsifiers, stabilizers, humectants and antioxidants may also be included, as well as agents imparting color or fragrance, if desired. Additionally, transdermal penetration enhancers may be employed in these topical formulations. Examples of such enhancers are found in U.S. Pat. Nos. 3,989,816 and 4,444,762.

Creams are preferably formulated from a mixture of mineral oil, self-emulsifying beeswax and water in which mixture of the active ingredient, dissolved in a small amount of an oil, such as almond oil, is admixed. A typical example of such a cream is one which includes approximately 40 parts water, approximately 20 parts beeswax, approximately 40 parts mineral oil and approximately 1 part almond oil.

Ointments may be formulated by mixing a solution of the active ingredient in a vegetable oil, such as almond oil, with warm soft paraffin and allowing the mixture to cool. A typical example of such an ointment is one that includes approximately 30% almond oil and approximately 70% white soft paraffin by weight.

The following examples are illustrative, but not limiting, of the method and compositions of the present invention. Other suitable modifications and adaptations of the variety of conditions and parameters normally encountered in clinical therapy and which are obvious to those skilled in the art are within the spirit and scope of the invention.

### EXAMPLE 1

### Methyl Tetrahydro-4-oxofuran-3-carboxylate

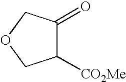

To a stirred slurry of sodium hydride (2.2 g, 55 mmol) in dry ether (40 mL) at room temperature (rt) was added methyl glycolate (4.5 g, 50 mmol) dropwise. The reaction mixture was stirred for ½ h, then it was concentrated under vacuo. To the solid was added methyl acrylate (5.2 g, 55 mmol) in DMSO (20 mL) at 0° C. and the mixture was stirred for 15 min, and the cool bath was removed and it was stirred for 45 min. The mixture was poured into 5% H₂SO₄ (60 mL), and it was extracted with ether (150 mL). The organic layer was dried, concentrated and purified by column chromatography to give 1.7 g (24%) of the title compound. ¹H NMR (CDCl₃): 4.51-4.40 (m, 2H), 4.03 (q, J=8.1 Hz, 2H), 3.80 (s, 3H), 3.54 (t, J=8.1 Hz, 1H).

### EXAMPLE 2

### 5,7-Dihydro-2-(methylthio)furo[3,4-d]pyrimidin-4-ol

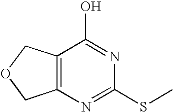

To a stirred solution of potassium hydroxide (1.97 g, 31.7 mmol) and 2-methyl-2-thiopseudourea sulfate (4.41 g, 31.7 mmol) in water (15 mL) was added dropwise of methyl tetrahydro-4-oxofuran-3-carboxylate (1.59 g, 15.9 mmol) over 5 min. The mixture was stirred at rt for 3 h and refluxed for 3 h. It was evaporated to dryness to give 4.1 g of crude product and used for next reaction without further purification.

### EXAMPLE 3

### 4-Chloro-5,7-dihydro-2-(methylthio)furo[3,4-d]pyrimidine

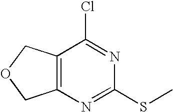

A mixture of the crude 5,7-dihydro-2-(methylthio)furo[3,4-d]pyrimidin-4-ol (4.1 g) in phosphorus oxychloride (8.0 mL) was refluxed for 1 h. The mixture was evaporated under vacuum and the residue was diluted with ethyl acetate (50 mL), washed with aqueous sodium bicarbonate (2×50 mL). The organic layer was dried, concentrated and evaporated, and the crude product was purified by column chromatography to give 438 mg (17%) of the title compound. ¹H NMR (CDCl₃): 5.11 (d, J=2.1 Hz, 2H), 5.01 (d, J=2.1 Hz, 2H), 2.58 (s, 3H).

### EXAMPLE 4

### 4-Chloro-5,7-dihydro-2-(methylsulfonyl)furo[3,4-d]pyrimidine

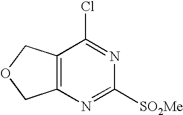

To a solution of 4-chloro-5,7-dihydro-2-(methylthio)furo[3,4-d]pyrimidine (110 mg, 0.55 mmol) in dichloromethane (4 mL) kept at 0° C. was added m-chloroperbenzoic acid (mCPBA, 305 mg, 1.36 mmol). The solution was warmed to rt and stirred for 1.5 h. It was diluted with dichloromethane (10 mL), washed with 50% sodium thiosulfate, saturated aqueous sodium bicarbonate and saline. The organic solution was evaporated to give the title compound. ¹H NMR (CDCl₃): 5.25 (bs, 2H), 5.21 (bs, 2H), 3.38 (s, 3H). MS. 234 (M+1).

### EXAMPLE 5

### 5,7-Dihydro-2-methylfuro[3,4-d]pyrimidin-4-ol

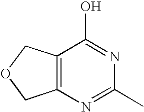

The title compound was prepared in a manner similar to example 2. From acetamidine hydrochloride (1.10 g, 11.7 mmol) and methyl tetrahydro-4-oxofuran-3-carboxylate (1.60 g, 11.7 mmol) was obtained 0.11 g (6%) of the title compound. ¹H NMR(CDCl₃): 5.08 (bs, 2H), 4.93 (bs, 2H), 2.52 (s, 3H).

### EXAMPLE 6

### 4-Chloro-5,7-dihydro-2-methylfuro[3,4-d]pyrimidine

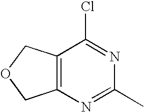

The title compound was prepared in a manner similar to example 3. From 5,7-dihydro-2-methylfuro[3,4-d]pyrimidin-4-ol (134 mg, 0.90 mmol) and phosphorus oxychloride (1.0 mL) was obtained 103 mg (67%) of the title compound. ¹H NMR (CDCl₃): 5.16 (bs, 2H), 5.07 (bs, 2H), 2.74 (s, 3H).

### EXAMPLE 7

### 5,7-Dihydro-N-(3,5-dimethoxyphenyl)-2-(methylthio)furo[3,4-d]pyrimidin-4-amine

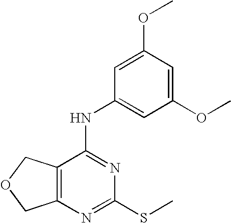

A mixture of 4-chloro-5,7-dihydro-2-(methylthio)furo[3,4-d]pyrimidine (50 mg, 0.25 mmol), and 3,5-dimethoxyaniline (76 mg, 0.50 mmol) in isopropanol (1.5 mL) was heated to 150° C. under microwave conditions for 40 min. The mixture was evaporated and the residue was purified by flash chromatography to give 12 mg (15%) of the title compound as a solid. ¹H NMR (CDCl₃): 6.58 (d, J=2.1 Hz, 2H), 6.39 (bs, 1H), 6.32 (t, J=2.4 Hz, 1H), 4.88 (bs, 2H), 4.76 (bs, 2H), 3.79 (s, 6H), 2.57 (s, 3H).

### EXAMPLE 8

### 5,7-Dihydro-N-(4-methoxyphenyl)-N-methyl-2-(methylthio)furo[3,4-d]pyrimidin-4-amine

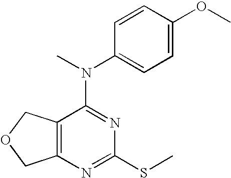

The title compound was prepared in a manner similar to example 7. From 4-chloro-5,7-dihydro-2-(methylthio)furo[3,4-d]pyrimidine (45 mg, 0.22 mmol) and N-methyl-p-anisidine (31 mg, 0.22 mmol) in isopropanol (1.5 mL) was obtained 41 mg (61%) of the title compound as a solid. ¹H NMR (CDCl₃): 7.13 (d, J=3.3 Hz, 2H), 6.91 (d, J=3.3 Hz, 2H), 4.73 (bs, 2H), 3.97 (bs, 2H), 3.84 (s, 3H), 3.46 (s, 3H), 2.58 (s, 3H).

### EXAMPLE 9

### 5,7-Dihydro-N-(3,5-dimethoxyphenyl)-furo[3,4-d]pyrimidin-4-amine

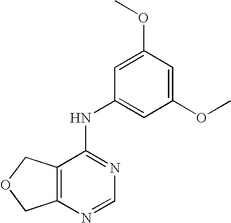

A solution of 5,7-dihydro-N-(3,5-dimethoxyphenyl)-2-(methylthio)furo[3,4-d]pyrimidin-4-amine (3 mg, 0.01 mmol) and Raney Ni in ethanol was hydrogenated under H₂ for 2 h. The mixture was filtered and solution was evaporated. The residue was purified by chromatography (EtOAc) to give 1 mg (30%) of the title compound. ¹H NMR (CDCl₃): 8.63 (s, 1H), 6.58 (d, J=2.1 Hz, 2H), 6.56 (bs, 1H), 6.34 (m, 1H), 4.95 (bs, 2H), 4.80 (bs, 2H), 3.80 (s, 6H). MS. 274 (M+1), 272 (M−1).

### EXAMPLE 10

### 5,7-Dihydro-N-(3-bromophenyl)-2-methylfuro[3,4-d]pyrimidin-4-amine

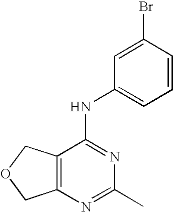

A solution of 4-chloro-5,7-dihydro-2-methylfuro[3,4-d]pyrimidine (16 mg, 0.10 mmol), and 3-bromoaniline (17.8 mg, 0.10 mmol) in DMF (1 mL) was heated at 150° C. for 2 h. It was diluted with EtOAc (5 mL) and washed with water (2×10 mL). The organic layer was dried, evaporated and the residue was purified by column chromatography (Hexane/EtOAc 1:3) to give 13 mg (45%) of the title compound as a solid. ¹H NMR (CDCl₃): 7.69 (s, 1H), 7.40-7.20 (m, 3H), 6.60 (bs, 1H), 4.94 (bs, 2H), 4.79 (bs, 2H), 2.61 (s, 3H).

### EXAMPLE 11

### 5,7-Dihydro-N-(3,5-dimethoxyphenyl)-2-methylfuro[3,4-d]pyrimidin-4-amine

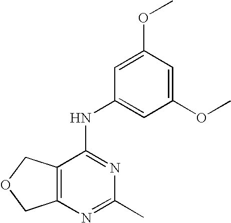

A solution of 4-chloro-5,7-dihydro-2-methylfuro[3,4-d]pyrimidine (25 mg, 0.15 mmol), 3,5-dimethoxyaniline (23 mg, 0.15 mmol) and 1 drop of concentrated HCl in isopropanol (1.5 mL) was refluxed for 1 h. The mixture was filtered and the precipitate was dried to give 15 mg (30%) of the title compound as a solid. ¹H NMR (CD₃OD): 6.85 (bs, 2H), 6.45 (bs, 1H), 5.09 (bs, 2H), 5.00 (bs, 2H), 3.82 (s, 6H), 2.69 (s, 3H).

### EXAMPLE 12

### 5,7-Dihydro-N-(3-methyl-1H-pyrazol-5-yl)-2-(methylthio)furo[3,4-d]pyrimidin-4-amine

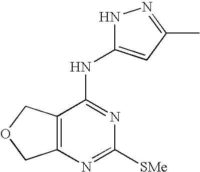

The title compound was prepared in a manner similar to previous example 7. From 4-chloro-5,7-dihydro-2-(methylthio)furo[3,4-d]pyrimidine (80 mg, 0.40 mmol), and 3-methyl-1H-pyrazol-5-amine (57 mg, 0.60 mmol) in isopropanol (1.5 mL) was obtained 15 mg (15%) of the title compound as a solid. ¹H NMR (CDCl₃): 6.79 (bs, 1H), 6.39 (bs, 1H), 4.90 (bs, 4H), 2.58 (s, 3H), 2.34 (s, 3H).

### EXAMPLE 13

### 5,7-Dihydro-N-(3-methyl-1H-pyrazol-5-yl)-2-(methylsulfonyl)furo[3,4-d]pyrimidin-4-amine

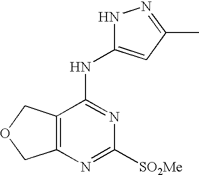

A solution of 4-chloro-5,7-dihydro-2-(methylsulfonyl)furo[3,4-d]pyrimidine (40 mg, 0.17 mmol), 3-methyl-1H-pyrazol-5-amine (17 mg, 0.17 mmol), diisopropyl ethylamine (22 mg, 0.17 mmol) and sodium iodide (26 mg, 0.17 mmol) in DMF (3 mL) was heated to 85° C. under argon for 5 h. It was cooled to rt and diluted with ethyl acetate (15 mL), washed with saline and evaporated. The residue was purified by flash chromatography to give 13 mg (26%) of the title compound as a solid. ¹H NMR (CDCl₃): 8.30 (bs, 1H), 6.37 (bs, 1H), 4.99 (bs, 2H), 4.87 (bs, 2H), 3.31 (s, 3H), 2.35 (s, 3H).

### EXAMPLE 14

### N-(4-(4-(3-Methyl-1H-pyrazol-5-ylamino)-5,7-dihydrofuro[3,4-d]pyrimidin-2-ylthio)phenyl)cyclopropanecarboxamide

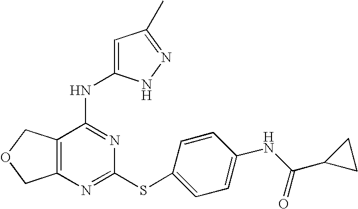

A mixture of 5,7-dihydro-N-(3-methyl-1H-pyrazol-5-yl)-2-(methylsulfonyl)furo[3,4-d]pyrimidin-4-amine (10 mg, 0.034 mmol) and N-(4-mercaptophenyl)cyclopropanecarboxamide (6.6 mg, 0.034 mmol) in t-butanol (2 mL) was heated to reflux under argon for 2 h. It was evaporated and the residue was purified by chromatography to give 7 mg of the title compound as a solid (50%). ¹H NMR (CD₃OD): 7.74 (d, J=9.0 Hz, 2H), 7.56 (d, J=9.0 Hz, 2H), 6.37 (s, 1H), 5.45 (bs, 1H), 4.94 (bs, 2H), 4.82 (bs, 2H), 2.06 (s, 3H), 1.80 (m, 1H), 0.90 (m, 2H), 0.85 (m, 2H). MS. 409 (M+1), 407 (M−1).

### EXAMPLE 15

### N-(4-(5,7-Dihydro-2-(methylsulfonyl)furo[3,4-d]pyrimidin-4-ylthio)phenyl)cyclopropanecarboxamide

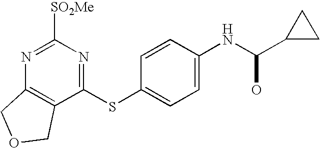

A solution of 4-chloro-5,7-dihydro-2-(methylsulfonyl)furo[3,4-d]pyrimidine (62 mg, 0.27 mmol) and N-(4-mercaptophenyl)cyclopropanecarboxamide (51 mg, 0.27 mmol) in t-butanol (3 mL) was heated in sealed tube to 90° C. under argon for 3 h. It was evaporated and the residue was purified by chromatography to give the 11 mg of the title compound as a solid (11%). ¹H NMR (CDCl₃): 7.81 (d, J=8.4 Hz, 2H), 7.67 (d, J=8.4 Hz, 2H), 5.20 (bs, 2H), 5.04 (bs, 2H), 3.20 (s, 3H), 1.70 (m, 1H), 1.30 (m, 2H), 1.10 (m, 2H).

### EXAMPLE 16

### N-(4-(2-(3-Methyl-1H-pyrazol-5-ylamino)-5,7-dihydrofuro[3,4-d]pyrimidin-4-ylthio)phenyl)cyclopropanecarboxamide

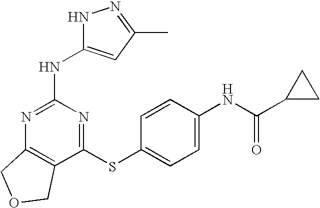

A solution of N-(4-(5,7-dihydro-2-(methylsulfonyl)furo[3,4-d]pyrimidin-4-ylthio)phenyl)cyclopropanecarboxamide (8 mg, 0.02 mmol), 3-methyl-1H-pyrazol-5-amine (3.9 mg, 0.04 mmol) and concentrated HCl (0.1 mL) in isopropanol (1 mL) was heated to 120° C. under microwave condition for 30 min. It was evaporated and the residue was purified by flash chromatography to give 2 mg (25%) of the title compound as a solid. ¹H NMR (CDCl₃): 7.62-7.58 (m, 4H), 7.25 (bs, 1H), 5.20 (s, 1H), 5.05 (bs, 2H), 5.02 (bs, 2H), 2.18 (s, 3H), 1.70 (m, 1H), 1.20 (m, 2H), 0.90 (m, 2H). MS. 409 (M+1), 407 (M−1).

### EXAMPLE 17

### (9H-Fluoren-9-yl)methyl 4-(3-methyl-1H-pyrazol-5-ylamino)-2-chloro-5H-pyrrolo[3,4-d]pyrimidine-6(7H)-carboxylate

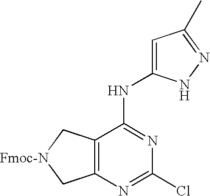

To a 10 mL microwave reaction flask charged with a magnetic stir bar, was added (9H-fluoren-9-yl)methyl 2,4-dichloro-5H-pyrrolo[3,4-d]pyrimidine-6(7H)-carboxylate (0.200 g, 0.485 mmol), isopropanol (2.40 mL), 3-methyl-1H-pyrazol-5-amine (0.094 g, 0.97 mmol) and p-toulenesulfonic acid (catalytic). The suspension was reacted at 130° C. for 20 minutes in a microwave set at 80 watts. The resulting precipitate was filtered and collected to give the crude product as a yellow solid. Purification by flash column chromatography (silica gel 12 g pre-packed column, gradient elution with EtOAc:Hexanes, 9:1 to 1:1) gave 0.035 g (15%) of the title compound as a white solid: ¹H-NMR (DMSO-d₆): 12.22 (br s, 1H), 10.29 (br s, 1H), 7.93-7.91 (m, 2H), 7.77-7.68 (m, 2H), 7.47-7.33 (m, 4H), 6.43 (m, 1H), 4.74 (m, 1H), 4.50-4.46 (m, 3H), 4.39-4.37 (m, 1H), 4.33-4.28 (m, 2H), 2.25 (d, J=2.1 Hz, 3H).

### EXAMPLE 18

### 6,7-Dihydro-N-(2,5-dimethoxyphenyl)-2-(methylthio)-5H-cyclopenta[d]pyrimidin-4-amine

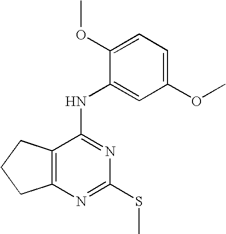

a) 6,7-Dihydro-2-(methylthio)-5H-cyclopenta[d]pyrimidin-4-ol. To a stirred solution of potassium hydroxide (12.0 g, 21.4 mmol) and 2-methyl-2-thiopseudourea sulfate (30.0 g, 10.7 mmol) in water (50 mL) was added methyl 2-oxocyclopentanecarboxylate (16.6 g, 10.6 mmol) dropwise over 5 minutes. The mixture was stirred at rt for 5 h and then to it was added a solution of potassium hydroxide (5.0 g, 80 mmol). It was heated to 100° C. for 0.5 h and cooled to rt. The mixture was filtered and the filtrate was neutralized with acetic acid to pH=5 to produce precipitates. The solid was collected by filtration and dried under vacuum to give 15.3 g (79%) of title compound without further purification.

b) 4-Chloro-6,7-dihydro-2-(methylthio)-5H-cyclopenta[d]pyrimidine. A mixture of 6,7-dihydro-2-(methylthio)-5H-cyclopenta[d]pyrimidin-4-ol (1.50 g, 8.24 mmol) in phosphorus oxychloride (4 mL) was heated to 90° C. for 10 min. It was cooled to rt and poured into ice water (50 mL). The precipitate was collected by filtration and dried to give 1.6 g of the title compound (100%). ¹H NMR (CDCl₃) 3.00 (t, J=7.5 Hz, 2H), 2.93 (t, J=7.5 Hz, 2H), 2.57 (s, 3H), 2.14 (m, 2H).

c) 6,7-Dihydro-N-(2,5-dimethoxyphenyl)-2-(methylthio)-5H-cyclopenta[d]pyrimidin-4-amine. The title compound was prepared in a manner similar to example 7. From 4-chloro-6,7-dihydro-2-(methylthio)-5H-cyclopenta[d]pyrimidine (235 mg, 1.07 mmol), and 2,5-dimethoxyaniline (153 mg, 1.1 mmol) was obtained 98 mg (31%) of the title compound as a solid. ¹H NMR (CDCl₃) 8.36 (d, J=3.0 Hz, 1H), 7.02 (bs, 1H), 6.81 (d, J=9.0 Hz, 1H), 6.89 (d, J=9.0 Hz, 1H), 6.54-6.50 (m, 1H), 3.88 (s, 3H), 3.81 (s, 3H), 2.91 (t, J=8.1 Hz, 2H), 2.78 (t, J=8.1 Hz, 2H), 2.63 (s, 3H), 2.15 (m, 2H).

### EXAMPLE 19

### N-(3-Bromophenyl)-6,7-dihydro-2-(methylthio)-5H-cyclopenta[d]pyrimidin-4-amine

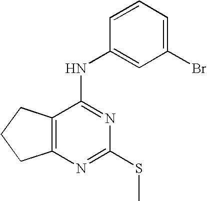

The title compound was prepared in a manner similar to example 7. From 4-chloro-6,7-dihydro-2-(methylthio)-5H-cyclopenta[d]pyrimidine (200 mg, 1.0 mmol) and 3-bromoaniline (172 mg, 1.0 mmol) was obtained 135 mg (40%) of the title compound as a solid. ¹H NMR (CDCl₃) 8.13 (bs, 1H), 7.40-7.37 (m, 1H), 7.20-7.17 (m, 2H), 6.20 (bs, 1H), 2.91 (t, J=7.8 Hz, 2H), 2.74 (t, J=7.5 Hz, 2H), 2.59 (s, 3H), 2.16 (m, 2H).

### EXAMPLE 20

### 6,7-Dihydro-N-(5-methyl-1H-pyrazol-3-yl)-2-(methylthio)-5H-cyclopenta[d]pyrimidin-4-amine

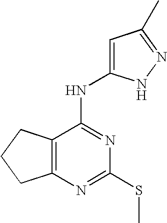

The title compound was prepared in a manner similar to example 7. From 4-chloro-6,7-dihydro-2-(methylthio)-5H-cyclopenta[d]pyrimidine (121 mg, 0.61 mmol), and 5-methyl-2H-pyrazol-3-amine (88 mg, 0.91 mmol) was obtained 6 mg (4%) of the title compound as a solid. ¹H NMR (CD₃OD) 6.51 (bs, 1H), 3.06 (t, J=7.5 Hz, 2H), 2.91 (t, J=7.5 Hz, 2H), 2.72 (s, 3H), 2.39 (s, 3H), 2.29 (m, 2H).

### EXAMPLE 21

### 6,7-Dihydro-N-(3,5-dimethoxyphenyl)-2-(methylthio)-5H-cyclopenta[d]pyrimidin-4-amine

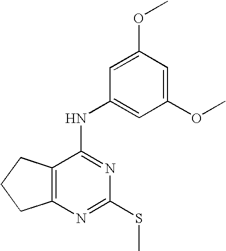

The title compound was prepared in a manner similar to example 7. From 4-chloro-6,7-dihydro-2-(methylthio)-5H-cyclopenta[d]pyrimidine (194 mg, 0.97 mmol), and 3,5-dimethoxyaniline (163 mg, 1.07 mmol) was obtained 135 g (44%) of the title compound as a solid. ¹H NMR (CDCl₃) 6.85 (bs, 2H), 6.21 (t, J=2.4 Hz, 1H), 6.20 (bs, 1H), 3.80 (s, 3H), 2.90 (t, J=7.8 Hz, 2H), 2.74 (t, J=7.8 Hz, 2H), 2.15 (m, 2H).

### EXAMPLE 22

### N-(4-(6,7-Dihydro-2-(methylthio)-5H-cyclopenta[d]pyrimidin-4-ylamino)phenyl)benzamide

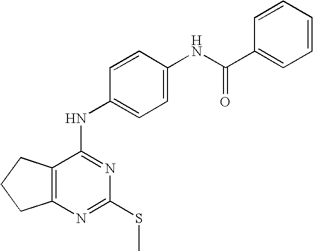

The title compound was prepared in a manner similar to example 7. From 4-chloro-6,7-dihydro-2-(methylthio)-5H-cyclopenta[d]pyrimidine (100 mg, 0.5 mmol) and N-(4-aminophenyl)benzamide (106 mg, 1 mmol) was obtained 75 mg (40%) of the title compound as a solid. ¹H NMR (CDCl₃) 7.89-7.85 (m, 3H), 7.61 (bs, 4H), 7.49-7.40 (m, 2H), 6.30 (bs, 1H), 2.88 (t, J=7.5 Hz, 2H), 2.70 (s, 3H), 2.54 (s, 3H), 2.12 (m, 2H).

### EXAMPLE 23

### 6,7-Dihydro-N-(4-methoxyphenyl)-N-methyl-2-(methylthio)-5H-cyclopenta[d]pyrimidin-4-amine

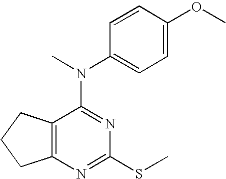

The title compound was prepared in a manner similar to example 7. From 4-chloro-6,7-dihydro-2-(methylthio)-5H-cyclopenta[d]pyrimidine (200 mg, 1.0 mmol) and N-methyl-4-methoxyaniline (151 mg, 1.1 mmol) was obtained 210 mg (69%) of the title compound as a solid. ¹H NMR (CDCl₃) 7.10 (d, J=9.0 Hz, 2H), 6.89 (d, J=9.0 Hz, 2H), 3.84 (s, 3H), 3.45 (s, 3H), 2.70 (t, J=8.1 Hz, 2H), 2.58 (s, 3H), 1.84 (m, 2H), 1.72 (m, 2H).

### EXAMPLE 24

### 6,7-Dihydro-N-(3,5-dimethoxyphenyl)-5H-cyclopenta[d]pyrimidin-4-amine

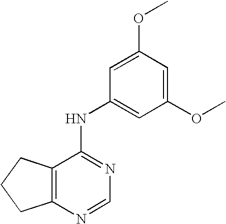

A mixture of 6,7-dihydro-N-(3,5-dimethoxyphenyl)-2-(methylthio)-5H-cyclopenta[d]pyrimidin-4-amine (40 mg, 0.13 mmol) and Raney Nickel (100 mg) in ethanol (5 mL) was refluxed for 4 h. It was filtered, the filtrate was evaporated and the crude product was purified by column chromatography to give the title compound (36 mg, 100%). ¹H NMR (CDCl₃) 8.59 (s, 1H), 6.84 (s, J=2.4 Hz, 2H), 6.23 (m, 2H), 3.81 (s, 3H), 2.96 (t, J=7.8 Hz, 2H), 2.76 (t, J=6.9 Hz, 2H), 2.16 (m, 2H).

### EXAMPLE 25

### 6,7-Dihydro-N-(2,5-dimethoxyphenyl)-5H-cyclopenta[d]pyrimidin-4-amine

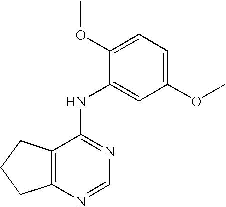

The title compound was prepared in a manner similar to example 24. From 6,7-dihydro-N-(2,5-dimethoxyphenyl)-2-(methylthio)-5H-cyclopenta[d]pyrimidin-4-amine (40 mg, 0.13 mmol) and Raney Nickel (300 mg) was obtained the title compound (22 mg, 63%) as a solid. ¹H NMR (CDCl₃) 8.62 (s, 1H), 8.41 (d, J=3.0 Hz, 1H), 7.00 (bs, 1H), 6.80 (s, J=8.7 Hz, 2H), 6.70 (m, 2H), 3.88 (s, 3H), 3.82 (s, 3H), 2.98 (t, J=8.1 Hz, 2H), 2.84 (t, J=8.1 Hz, 2H), 2.17 (m, 2H).

### EXAMPLE 26

### N-(4-(6,7-Dihydro-5H-cyclopenta[d]pyrimidin-4-ylamino)phenyl)benzamide

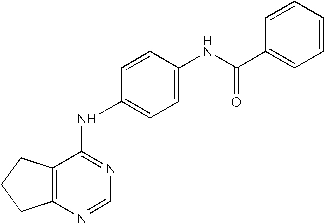

The title compound was prepared in a manner similar to example 24. From N-(4-(6,7-dihydro-2-(methylthio)-5H-cyclopenta[d]pyrimidin-4-ylamino)phenyl)benzamide (42 mg, 0.11 mmol) and Raney Nickel (300 mg) was obtained 27 mg (75%) of the title compound. ¹H NMR (CDCl₃) 8.80 (s, 1H), 8.00-7.90 (m, 3H), 7.70-7.45 (m, 7H), 6.31 (s, 1H), 2.97 (t, J=7.8 Hz, 2H), 2.77 (t, J=7.2 Hz, 2H), 2.22-2.10 (m, 2H).

### EXAMPLE 27

### 6,7-Dihydro-N-(3,5-dimethoxyphenyl)-2-(methylsulfinyl)-5H-cyclopenta[d]pyrimidin-4-amine

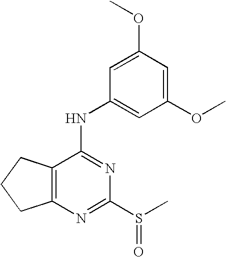

To a solution of 6,7-dihydro-N-(3,5-dimethoxyphenyl)-2-(methylthio)-5H-cyclopenta[d]pyrimidin-4-amine (20 mg, 0.06 mmol) in dichloromethane (2 mL) kept at 0° C. was added m-CPBA (0.06 mmol), and it was warmed to rt and stirred for 1 h. It was diluted with ethyl acetate and washed with water, and the organic layer was dried and concentrated to give the title compound. ¹H NMR (CDCl₃) 6.87 (bs, 2H), 6.68 (bs, 1H), 6.05 (bs, 1H), 3.80 (s, 6H), 3.04 (t, J=7.8 Hz, 2H), 2.92 (t, 3H), 2.79 (t, J=6.9 Hz, 2H), 2.17 (m, 2H). MS. 334 (M+1), 332 (M−1).

### EXAMPLE 28

### [2-(4-Methyl-piperazin-1-yl)-6,7-dihydro-5H-pyrrolo[3,4-d]pyrimidin-4-yl]-(5-methyl-2H-pyrazol-3-yl)-amine

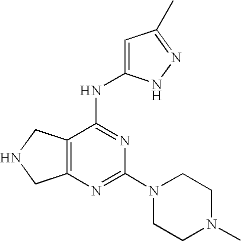

To an oven dried sealed reaction vessel charged with a magnetic stir bar at rt was added 2-chloro-4-(5-methyl-2H-pyrazol-3-ylamino)-5,7-dihydro-pyrrolo[3,4-d]pyramid-dine-6-carboxylic acid 9H-fluoren-9-ylmethyl ester (0.030 g, 0.063 mmol), isopropanol (0.32 mL) and N-methylpiperazine (distilled, 0.051 g, 0.51 mmol). The yellow suspension was heated at 80° C. for 2 h. The resulting suspension was concentrated by rotary evaporation to give the crude product as a yellow solid. It was purified by flash column chromatography (silica gel 4 g pre-packed column, gradient elution with CH₂Cl₂:MeOH, 96:4 to MeOH), gave 0.002 g (6%) of the title compound as a white solid. ¹H NMR (CD₃OD) 6.24 (s, 1H), 3.80-3.76 (m, 4H), 3.35-3.24 (m, 4H), 2.53-2.48 (m, 4H), 2.27 (m, 3H), 2.33 (d, J=2.7 Hz, 3H).

### EXAMPLE 29

### (9H-Fluoren-9-yl)methyl 2-chloro-4-((4-(cyclopropanecarboxamido)phenyl)sulfanyl)-5H-pyrrolo[3,4-d]pyrimidine-6(7H)-carboxylate

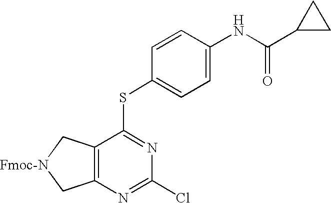

To an oven dried sealed reaction vessel charged with a magnetic stir bar at rt was added (9H-fluoren-9-yl)methyl 2,4-dichloro-5H-pyrrolo[3,4-d]pyrimidine-6(7H)-carboxylate (0.200 g, 0.485 mmol), THF (2.4 mL) and N-(4-mercaptophenyl)cyclopropanecarboxamide (0.094 g, 0.49 mmol). The yellow suspension was stirred at rt for 20 h and then concentrated by rotary evaporation to give 0.270 g (97%) of the title compound as a yellow solid. ¹H NMR (DMSO-d₆) 10.16 (br s, 1H), 7.92 (d, J=7.4 Hz, 2H), 7.70 (d, J=7.1 Hz, 2H), 7.49-7.33 (m, 6H), 7.21 (d, J=8.5 Hz, 2H), 4.70-4.62 (m, 4H), 4.42-4.35 (m, 3H), 1.74 (m, 1H), 0.78-0.76 (m, 4H).

### EXAMPLE 30

### (9H-Fluoren-9-yl)methyl 4-(3-(dimethylamino)-5-methoxyphenylamino)-2-chloro-5H-pyrrolo[3,4-d]pyrimidine-6(7H)-carboxylate

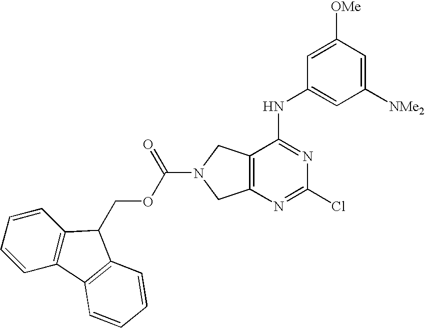

To an oven-dried round bottom reaction flask charged with a magnetic stir bar at rt under argon was added (9H-fluoren-9-yl)methyl 2,4-dichloro-5H-pyrrolo[3,4-d]pyrimidine-6(7H)-carboxylate (0.062 g, 0.150 mmol), isopropanol (0.75 mL), 5-methoxy-3-(dimethylamino)-aniline (0.025 g, 0.15 mmol) and concentrated HCl (3 drops). The brown suspension was heated at 85° C. for 3 h, and then cooled to rt. The resulting mixture was filtered to remove the solids. The filtrate was purified by flash column chromatography (silica gel 4 g pre-packed column, gradient elution with EtOAc:Hexanes, 1:4 to 1:1) to give 0.004 g (5%) of the title compound as an white solid: ES MS (C₃₀H₂₈ClN₅O₃): 542 [M+H]⁺ (100%), 544 [M+H]⁺ (35%).

### EXAMPLE 31

### (9H-Fluoren-9-yl)methyl 4-(N-(4-methoxyphenyl)-N-methylamino)-2-chloro-5H-pyrrolo[3,4-d]pyrimidine-6(7H)-carboxylate

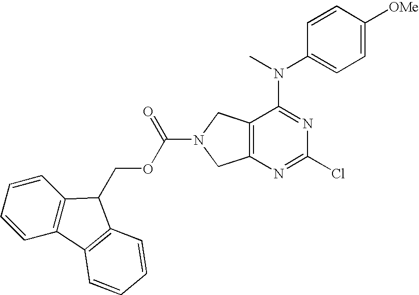

To an oven-dried sealed reaction flask charged with a magnetic stir bar at rt under argon was added (9H-fluoren-9-yl)methyl 2,4-dichloro-5H-pyrrolo[3,4-d]pyrimidine-6(7H)-carboxylate (0.062 g, 0.150 mmol), isopropanol (0.75 mL), N-methyl-p-anisidine (0.021 g, 0.15 mmol) and p-toluenesulfonic acid (catalytic). The yellow suspension was heated at 90° C. for 2 h, and then cooled to rt. The resulting mixture was filtered to remove the solids. The filtrate was purified by flash column chromatography (silica gel 4 g pre-packed column, elution with EtOAc:Hexanes, 1:1) gave 0.002 g (2%) of the title compound as an white solid: ES MS (C₂₉H₂₅ClN₄O₃): 513 [M+H]⁺ (100%), 515 [M+H]⁺ (35%).

### EXAMPLE 32

### 5,7-Dihydro-N-(3-methyl-1H-pyrazol-5-yl)-2-(4-methylpiperazin-1-yl)furo[3,4-d]pyrimidin-4-amine

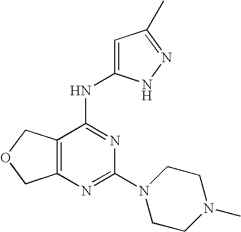

The title compound was prepared in a manner similar to example 14. From 5,7-dihydro-N-(5-methyl-1H-pyrazol-3-yl)-2-(methylsulfonyl)furo[3,4-d]pyrimidin-4-amine and N-methylpiperazine was obtained 10 mg (62%) of the title compound. ¹H NMR (CDCl₃): 6.75 (s, 1H), 6.25 (s, 1H), 4.88 (bs, 2H), 4.82 (bs, 2H), 3.86 (t, J=5.0 Hz, 4H), 2.53 (t, J=5.0 Hz, 4H), 2.38 (s, 3H), 2.32 (s, 3H).

### EXAMPLE 33

### Identification Of 5,7-Dihydro-N-(3,5-dimethoxyphenyl)-2-(methylthio)furo[3,4-d]pyrimidin-4-amine And Analogs As Caspase Cascade Activators And Inducers Of Apoptosis In Solid Tumor Cells

Human breast cancer cell line T-47D, human hepatocellular carcinoma cell line SNU398 and human colon carcinoma cell line HCT116 were grown according to media component mixtures designated by American Type Culture Collection+10% FCS (Invitrogen Corporation), in a 5% CO₂-95% humidity incubator at 37° C. T-47D and H1299 cells were maintained at a cell density between 50 and 80% confluency at a cell density of 0.1 to 0.6×10⁶ cells/mL. Cells were harvested at 600×g and resuspended at 0.65×10⁶ cells/mL into appropriate media+10% FCS. An aliquot of 22.5 μL of cells was added to a well of a 384-well microtiter plate containing 2.5 μL of a 10% DMSO in RPMI-1640 media solution containing 0.16 to 100 μM of 5,7-dihydro-N-(3,5-dimethoxyphenyl)-2-(methylthio)furo[3,4-d]pyrimidin-4-amine or other test compound (0.016 to 10 μM final). An aliquot of 22.5 μL of cells was added to a well of a 384-well microtiter plate containing 2.5 μL of a 10% DMSO in RPMI-1640 media solution without test compound as the control sample. The samples were mixed by agitation and then incubated at 37° C. for 24 or 48 h in a 5% CO₂-95% humidity incubator. After incubation, the samples were removed from the incubator and 25 μL of a solution containing 14 μM of N-(Ac-DEVD)-N′-ethoxycarbonyl-R110 (SEQ ID No.:1) fluorogenic substrate (Cytovia, Inc.; WO99/18856), 20% sucrose (Sigma), 20 mM DTT (Sigma), 200 mM NaCl (Sigma), 40 mM Na PIPES buffer pH 7.2 (Sigma), and 500 g/mL lysolecithin (Calbiochem) was added. The samples were mixed by agitation and incubated at room temperature. Using a fluorescent plate reader (Model SPECTRAfluor Plus, Tecan), an initial reading (T=0) was made approximately 1-2 min after addition of the substrate solution, employing excitation at 485 nm and emission at 530 nm, to determine the background fluorescence of the control sample. After the 3 h incubation, the samples were read for fluorescence as above (T=3 h).

Calculation:

The Relative Fluorescence Unit values (RFU) were used to calculate the sample readings as follows:
RFU_{(T=3h)}−Control RFU_{(T=0)}=Net RFU_{(T=3h)}

The activity of caspase cascade activation was determined by the ratio of the net RFU value for 5,7-dihydro-N-(3,5-dimethoxyphenyl)-2-(methylthio)furo[3,4-d]pyrimidin-4-amino or other test compound to that of control samples. The EC₅₀ (nM) was determined by a sigmoidal dose-response calculation (Prism 3.0, GraphPad Software Inc.).

The caspase activation potency (EC₅₀) are summarized in Table I:

TABLE I
Caspase Activation Potency

| | EC₅₀ (nM) | | |
|---|---|---|---|
| Example | T-47D | HCT116 | SNU398 |
| 7 | 61 | 5504 | 4711 |
| 8 | 3185 | 5147 | 2557 |
| 9 | 1187 | >10000 | >10000 |
| 10 | >10000 | >10000 | >10000 |
| 11 | 1286 | >10000 | >10000 |
| 12 | >10000 | >10000 | >10000 |
| 13 | >10000 | >10000 | >10000 |
| 14 | 2738 | 2422 | 1361 |
| 15 | 14082 | 14223 | 9069 |
| 16 | >10000 | >10000 | >10000 |
| 17 | >10000 | >10000 | >10000 |
| 18 | 134 | 286 | 1322 |
| 19 | >10000 | >10000 | >10000 |
| 20 | >10000 | 10604 | 2693 |
| 21 | 63 | >10000 | 2556 |
| 22 | >10000 | >10000 | >10000 |
| 23 | 31 | ND | ND |
| 24 | 987 | >10000 | >10000 |
| 25 | 260 | >10000 | >10000 |
| 26 | >10000 | >10000 | >10000 |
| 27 | 164 | >10000 | >10000 |
| 28 | >10000 | >10000 | >10000 |
| 29 | >10000 | >10000 | >10000 |
| 30 | 1569 | >10000 | >10000 |
| 31 | >10000 | >10000 | 5000 |
| 32 | >10000 | >10000 | >10000 |

ND, not determined.

Thus, 5,7-dihydro-N-(3,5-dimethoxyphenyl)-2-(methylthio)furo[3,4-d]pyrimidin-4-amine (Example 7) and analogs are identified as potent caspase cascade activators and inducers of apoptosis in solid tumor cells.

Several compounds were also tested in other cancer cells lines (24 h assay), including human breast cancer cell line T-47D, human lung cancer cell line H1299, human hepatocellular carcinoma cell line SNU398 and breast cancer cell line SKBR3, and the data are summarized in Table II.

TABLE II
Caspase Activation Potency

EC₅₀ (nM) (24 h)

| Example | T47D | SNU398 | H1299 | SKBR3 |
|---|---|---|---|---|
| 7 | 54 | >10000 | ND | 118 |
| 18 | 82 | >10000 | 934 | ND |
| 23 | 32 | 35 | 43 | ND |
| 25 | 244 | >10000 | >10000 | 355 |

ND, not determined.

Thus, these data indicated that 5,7-dihydro-N-(3,5-dimethoxyphenyl)-2-(methylthio)furo[3,4-d]pyrimidin-4-amine (Example 7) and analogs are potent caspase cascade activators and inducers of apoptosis in several tumor cells.

### EXAMPLE 34

### Identification Of 5,7-Dihydro-N-(3,5-dimethoxyphenyl)-2-(methylthio)furo[3,4-d]pyrimidin-4-amine And Analogs As Antineoplastic Compound That Inhibits Cell Proliferation (GI₅₀)

T-47D, MX-1, SNU398 and HCT116 cells were grown and harvested as in Example 33. An aliquot of 90 μL of cells (4.4×10⁴ cells/mL) was added to a well of a 96-well microtiter plate containing 5 μL of a 10% DMSO in RPMI-1640 media solution containing 10 nM to 100 μM of 5,7-dihydro-N-(3,5-dimethoxyphenyl)-2-(methylthio)furo[3,4-d]pyrimidin-4-amine (1 nM to 10 μM final) or related compounds. An aliquot of 45 μL of cells was added to a well of a 96-well microtiter plate containing 5 μL of a 10% DMSO in RPMI-1640 media solution without compound as the control sample for maximal cell proliferation (L_{Max}). The samples were mixed by agitation and then incubated at 37° C. for 48 h in a 5% CO₂-95% humidity incubator. After incubation, the samples were removed from the incubator and 25 μL of CellTiter-Glo™ reagent (Promega) was added. The samples were mixed by agitation and incubated at room temperature for 10-15 min. Plates were then read using a luminescent plate reader (Model SPECTRAfluor Plus, Tecan) to give Lₜₑₛₜ values.

Baseline for GI₅₀ (dose for 50% inhibition of cell proliferation) of initial cell numbers was determined by adding an aliquot of 45 μL of cells or 45 μL of media, respectively, to wells of a 96-well microtiter plate containing 5 μL of a 10% DMSO in RPMI-1640 media solution. The samples were mixed by agitation and then incubated at 37° C. for 0.5 h in a 5% CO₂-95% humidity incubator. After incubation, the samples were removed from the incubator and 25 μL of CellTiter-Glo™ reagent (Promega) was added. The samples were mixed by agitation and incubated at 37° C. for 10-15 min at room temperature in a 5% CO₂-95% humidity incubator. Fluorescence was read as above, (L_{Start}) defining luminescence for initial cell number used as baseline in GI₅₀ determinations.

Calculation:

GI₅₀ (dose for 50% inhibition of cell proliferation) is the concentration where [(L_{Test}−L_{Start})/(L_{Max}−L_{Start})]=0.5.

The GI₅₀ (nM) are summarized in Table III:

TABLE III
GI₅₀ in Cancer Cells

GI₅₀ (nM)

| Example | T47D | MX1 | SNU398 | HCT116 |
|---|---|---|---|---|
| 7 | 183 | 27 | 3736 | 3000 |
| 18 | 21 | 200 | >10000 | >10000 |
| 23 | 46 | 50 | ND | ND |
| 24 | 5000 | 5000 | >10000 | >10000 |
| 25 | 1000 | 890 | >10000 | >10000 |
| 27 | 500 | 600 | >10000 | >10000 |

ND, not determined.

Thus, 5,7-dihydro-N-(3,5-dimethoxyphenyl)-2-(methylthio)furo[3,4-d]pyrimidin-4-amine (Example 7) and analogs are identified as antineoplastic compound that inhibits cell proliferation.

Compound of Example 23 were also tested in additional cancer cell lines, including human breast cancer cell lines MDAMB435, human sarcoma cell line MES-SA and multi-drug resistant (MDR) human sarcoma cell line MES-SA/ADR, murine leukemia cell line P388 and multi-drug resistant (MDR) murine leukemia cell line P388ADR, and the data are summarized in Table IV.

TABLE IV
GI₅₀ in Multi-Cancer Cells

| | GI₅₀ (nM) | | | | |
|---|---|---|---|---|---|
| | | | MES- | | |
| Example | MDAMB435 | MES-SA | SA/ADR | P388 | P388ADR |
| 23 | 4 | 26 | 26 | 14 | 21 |

Thus, 6,7-dihydro-N-(4-methoxyphenyl)-N-methyl-2-(methylthio)-5H-cyclopenta[d]pyrimidin-4-amine (Example 23) is identified as antineoplastic compound that inhibits cell proliferation in several cancer cell lines. More importantly, compound of example 23 was found to have similar activity against MES-SA and its corresponding multi-drug resistant cell MES-SA/ADR, as well as P388 and its corresponding multi-drug resistant cell P388/ADR.

Having now fully described this invention, it will be understood by those of ordinary skill in the art that the same can be performed within a wide and equivalent range of conditions, formulations and other parameters without affecting the scope of the invention or any embodiment thereof. All patents, patent applications, and publications cited herein are fully incorporated by reference herein in their entirety.

1. A method of treating a disorder responsive to the induction of apoptosis in an animal suffering therefrom, comprising administering to an animal in need of such treatment an effective amount of a compound having the Formula I: 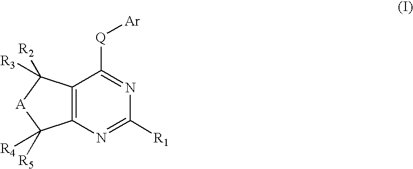
or a pharmaceutically acceptable salt or prodrug or tautomer thereof, wherein:
Ar is optionally substituted aryl or optionally substituted heteroaryl;
A is O, S(O)ₙ, NR₇, or CR₉R₁₀, wherein n is 0, 1 or 2, R₇ is R₈, COR₈, CONHR₈, COOR₈, or SO₂R₈, wherein R₈-R₁₀ independently are hydrogen, C₁₋₁₀ alkyl, haloalkyl, aryl, carbocyclic, a heterocyclic group, a heteroaryl group, alkenyl, alkynyl, arylalkyl, arylalkenyl, arylalkynyl, heteroarylalkyl, heteroarylalkenyl, heteroarylalkynyl, carbocycloalkyl, heterocycloalkyl, hydroxyalkyl, aminoalkyl, or carboxyalkyl;
Q is O, S or NR₆, wherein R₆ is hydrogen or an optionally substituted alkyl;
R₁ is hydrogen, halo, optionally substituted amino, alkylamino, arylamino, alkylthiol, alkylsulfone, alkylsulfoxide, arylthiol, alkoxy, or aryloxy, optionally substituted C₁₋₁₀ alkyl, haloalkyl, aryl, carbocyclic, a heterocyclic group, a heteroaryl group, alkenyl, alkynyl, arylalkyl, arylalkenyl, arylalkynyl, heteroarylalkyl, heteroarylalkenyl, heteroarylalkynyl, carbocycloalkyl, heterocycloalkyl, hydroxyalkyl, aminoalkyl, aminoaryl, carboxyalkyl, nitro, cyano, acylamido, hydroxy, acyloxy, azido, carboxy, or carbonylamido; and
R₂-R₅ independently are hydrogen, halo, amino, alkoxy, C₁₋₁₀ alkyl, haloalkyl, aryl, carbocyclic, a heterocyclic group, a heteroaryl group, alkenyl, alkynyl, arylalkyl, arylalkenyl, arylalkynyl, heteroarylalkyl, heteroarylalkenyl, heteroarylalkynyl, carbocycloalkyl, heterocycloalkyl, hydroxyalkyl, aminoalkyl, carboxyalkyl, nitro, cyano, acylamido, hydroxy, thiol, acyloxy, azido, carboxy, methylenedioxy, carbonylamido or alkylthiol, alkylsulfone, alkylsulfoxide.

2. The method of claim 1, wherein said animal is a mammal.

3. The method of claim 1, wherein Ar is optionally substituted pyrrazol, triaziol, thiadiazol, imidazole, oxazol, thiazol, phenyl, pyridyl, or pyrimidyl.

4. The method of claim 1, wherein R₁ is hydrogen, halo, optionally substituted amino, alkylamino, or arylamino, optionally substituted alkoxy, aryloxy, optionally substituted alkylthiol, alkylsulfone, alkylsulfoxide, or arylthiol, optionally substituted aryl, optionally substituted heteroaryl, or optionally substituted C₁₋₁₀ alkyl.

5. The method of claim 1, wherein Q is NH.

6. The method of claim 1, wherein A is O.

7. The method of claim 1, wherein A is S, SO, or SO₂.

8. The method of claim 1, wherein A is NR₇, R₇ is R₈, COR₈, CONHR₈, COOR₈, or SO₂R₈, and R₈ is hydrogen, C₁₋₁₀ alkyl, haloalkyl, aryl, carbocyclic, a heterocyclic group, a heteroaryl group, alkenyl, alkynyl, arylalkyl, arylalkenyl, arylalkynyl, heteroarylalkyl, heteroarylalkenyl, heteroarylalkynyl, carbocycloalkyl, heterocycloalkyl, hydroxyalkyl, aminoalkyl, or carboxyalkyl.

9. The method of claim 1, wherein A is CR₉R₁₀, wherein R₉-R₁₀ independently are hydrogen, C₁₋₁₀ alkyl, haloalkyl, aryl, carbocyclic, a heterocyclic group, a heteroaryl group, alkenyl, alkynyl, arylalkyl, arylalkenyl, arylalkynyl, heteroarylalkyl, heteroarylalkenyl, heteroarylalkynyl, carbocycloalkyl, heterocycloalkyl, hydroxyalkyl, aminoalkyl, or carboxyalkyl.

10. A method of treating a disorder responsive to the induction of apoptosis in an animal suffering therefrom, comprising administering to an animal in need of such treatment an effective amount of a compound having the Formula II: 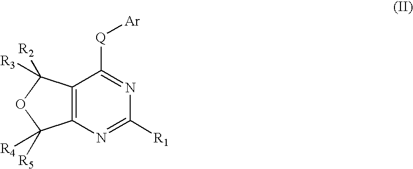
or a pharmaceutically acceptable salt or prodrug or tautomer thereof, wherein:
Ar is optionally substituted aryl or optionally substituted heteroaryl;
Q is O, S or NR₆, wherein R₆ is hydrogen or an optionally substituted alkyl;
R₁ is hydrogen, halo, optionally substituted amino, alkylamino, arylamino, alkylthiol, alkylsulfone, alkylsulfoxide, arylthiol, alkoxy, or aryloxy, optionally substituted C₁₋₁₀ alkyl, haloalkyl, aryl, carbocyclic, a heterocyclic group, a heteroaryl group, alkenyl, alkynyl, arylalkyl, arylalkenyl, arylalkynyl, heteroarylalkyl, heteroarylalkenyl, heteroarylalkynyl, carbocycloalkyl, heterocycloalkyl, hydroxyalkyl, aminoalkyl, aminoaryl, carboxyalkyl, nitro, cyano, acylamido, hydroxy, acyloxy, azido, carboxy, or carbonylamido; and
R₂-R₅ independently are hydrogen, halo, amino, alkoxy, C₁₋₁₀ alkyl, haloalkyl, aryl, carbocyclic, a heterocyclic group, a heteroaryl group, alkenyl, alkynyl, arylalkyl, arylalkenyl, arylalkynyl, heteroarylalkyl, heteroarylalkenyl, heteroarylalkynyl, carbocycloalkyl, heterocycloalkyl, hydroxyalkyl, aminoalkyl, carboxyalkyl, nitro, cyano, acylamido, hydroxy, thiol, acyloxy, azido, carboxy, methylenedioxy, carbonylamido or alkylthiol, alkylsulfone, alkylsulfoxide.

11. The method of claim 10, wherein Ar is optionally substituted pyrrazol, triaziol, thiadiazol, imidazole, oxazol, thiazol, phenyl, pyridyl, or pyrimidyl.

12. The method of claim 10, wherein R₁ is hydrogen, halo, optionally substituted amino, alkylamino, or arylamino, optionally substituted alkoxy, aryloxy, optionally substituted alkylthiol, alkylsulfone, alkylsulfoxide, or arylthiol, optionally substituted aryl, optionally substituted heteroaryl, or optionally substituted C₁₋₁₀ alkyl.

13. The method of claim 10, wherein Q is NH.

14. A method of treating a disorder responsive to the induction of apoptosis in an animal suffering therefrom, comprising administering to an animal in need of such treatment an effective amount of a compound having the Formula V: 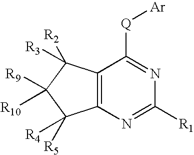
or a pharmaceutically acceptable salt or prodrug or tautomer thereof, wherein:
Ar is optionally substituted aryl or optionally substituted heteroaryl;
Q is O, S or NR₆, wherein R₆ is hydrogen or an optionally substituted alkyl;
R₁ is hydrogen, halo, optionally substituted amino, alkylamino, arylamino, alkylthiol, alkylsulfone, alkylsulfoxide, arylthiol, alkoxy, or aryloxy, optionally substituted C₁₋₁₀ alkyl, haloalkyl, aryl, carbocyclic, a heterocyclic group, a heteroaryl group, alkenyl, alkynyl, arylalkyl, arylalkenyl, arylalkynyl, heteroarylalkyl, heteroarylalkenyl, heteroarylalkynyl, carbocycloalkyl, heterocycloalkyl, hydroxyalkyl, aminoalkyl, aminoaryl, carboxyalkyl, nitro, cyano, acylamido, hydroxy, acyloxy, azido, carboxy, or carbonylamido; and
R₂-R₅ and R₉-R₁₀ independently are hydrogen, halo, amino, alkoxy, C₁₋₁₀ alkyl, haloalkyl, aryl, carbocyclic, a heterocyclic group, a heteroaryl group, alkenyl, alkynyl, arylalkyl, arylalkenyl, arylalkynyl, heteroarylalkyl, heteroarylalkenyl, heteroarylalkynyl, carbocycloalkyl, heterocycloalkyl, hydroxyalkyl, aminoalkyl, carboxyalkyl, nitro, cyano, acylamido, hydroxy, thiol, acyloxy, azido, carboxy, methylenedioxy, carbonylamido or alkylthiol, alkylsulfone, alkylsulfoxide.

15. The method of claim 14, wherein Ar is optionally substituted pyrrazol, triaziol, thiadiazol, imidazole, oxazol, thiazol, phenyl, pyridyl, or pyrimidyl.

16. The method of claim 14, wherein R₁ is hydrogen, halo, optionally substituted amino, alkylamino, or arylamino, optionally substituted alkoxy, aryloxy, optionally substituted alkylthiol, alkylsulfone, alkylsulfoxide, or arylthiol, optionally substituted aryl, optionally substituted heteroaryl, or optionally substituted C₁₋₁₀ alkyl.

17. The method of claim 14, wherein Q is NR₆, and R₆ is hydrogen or an optionally substituted alkyl.

18. A method of treating a disorder responsive to the induction of apoptosis in an animal suffering therefrom, comprising administering to an animal in need of such treatment an effective amount of a compound selected from the group consisting of:
5,7-Dihydro-N-(3,5-dimethoxyphenyl)-2-(methylthio)furo[3,4-d]pyrimidin-4-amine;
5,7-Dihydro-N-(4-methoxyphenyl)-N-methyl-2-(methylthio)furo[3,4-d]pyrimidin-4-amine;
5,7-Dihydro-N-(3,5-dimethoxyphenyl)-furo[3,4-d]pyrimidin-4-amine;
5,7-Dihydro-N-(3,5-dimethoxyphenyl)-2-methylfuro[3,4-d]pyrimidin-4-amine;
N-(4-(4-(3-Methyl-1H-pyrazol-5-ylamino)-5,7-dihydrofuro[3,4-d]pyrimidin-2-ylthio)phenyl)cyclopropanecarboxamide;
N-(4-(5,7-Dihydro-2-(methylsulfonyl)furo[3,4-d]pyrimidin-4-ylthio)phenyl)cyclopropanecarboxamide;
6,7-Dihydro-N-(2,5-dimethoxyphenyl)-2-(methylthio)-5H-cyclopenta[d]pyrimidin-4-amine;
6,7-Dihydro-N-(5-methyl-1H-pyrazol-3-yl)-2-(methylthio)-5H-cyclopenta[d]pyrimidin-4-amine;
6,7-Dihydro-N-(3,5-dimethoxyphenyl)-2-(methylthio)-5H-cyclopenta[d]pyrimidin-4-amine;
6,7-Dihydro-N-(4-methoxyphenyl)-N-methyl-2-(methylthio)-5H-cyclopenta[d]pyrimidin-4-amine;
6,7-Dihydro-N-(3,5-dimethoxyphenyl)-5H-cyclopenta[d]pyrimidin-4-amine;
6,7-Dihydro-N-(2,5-dimethoxyphenyl)-5H-cyclopenta[d]pyrimidin-4-amine;
6,7-Dihydro-N-(3,5-dimethoxyphenyl)-2-(methylsulfinyl)-5H-cyclopenta[d]pyrimidin-4-amine;
(9H-Fluoren-9-yl)methyl 4-(3-(dimethylamino)-5-methoxyphenylamino)-2-chloro-5H-pyrrolo[3,4-d]pyrimidine-6(7H)-carboxylate;
(9H-Fluoren-9-yl)methyl 4-(N-(4-methoxyphenyl)-N-methylamino)-2-chloro-5H-pyrrolo[3,4-d]pyrimidine-6(7H)-carboxylate; and
5,7-Dihydro-N-(3-methyl-1H-pyrazol-5-yl)-2-(4-methylpiperazin-1-yl)furo[3,4-d]pyrimidin-4-amine;
or a pharmaceutically acceptable salt or prodrug thereof.

19. The method of claim 1, wherein said disorder is cancer.

20-21. (canceled)

22. The method of claim 19, further comprising administering at least one known cancer chemotherapeutic agent, or a pharmaceutically acceptable salt of said agent.

23. The method according to claim 19, wherein said compound is administered together with at least one compound selected from the group consisting of busulfan, cis-platin, mitomycin C, carboplatin, colchicine, vinblastine, paclitaxel, docetaxel, camptothecin, topotecan, doxorubicin, etoposide, 5-azacytidine, 5-fluorouracil, methotrexate, 5-fluoro-2′-deoxy-uridine, ara-C, hydroxyurea, thioguanine, melphalan, chlorambucil, cyclophosamide, ifosfamide, vincristine, mitoguazone, epirubicin, aclarubicin, bleomycin, mitoxantrone, elliptinium, fludarabine, octreotide, retinoic acid, tamoxifen, Herceptin®, Rituxan®, arsenic trioxide, gemcitabine, doxazosin, terazosin, tamsulosin, CB-64D, CB-184, haloperidol, lovastatin, simvastatin, pravastatin, fluvastatin, atorvastatin, cerivastatin, amprenavir, abacavir, CGP-73547, CGP-61755, DMP-450, indinavir, nelfinavir, tipranavir, ritonavir, saquinavir, ABT-378, AG 1776, BMS-232,632, bexarotene, tretinoin, 13-cis-retinoic acid, 9-cis-retinoic acid, α-difluoromethylornithine, ILX23-7553, fenretinide, N-4-carboxyphenyl retinamide, lactacystin, MG-132, PS-341, Gleevec®, ZD1839 (Iressa), SH268, genistein, CEP2563, SU6668, SU11248, EMD121974, R115777, SCH66336, L-778,123, BAL9611, TAN-1813, flavopiridol, UCN-01, roscovitine, olomoucine, celecoxib, valecoxib, rofecoxib and alanosine.

24. The method of claim 19, further comprising treating said animal with radiation-therapy.

25. The method of claim 19, wherein said compound is administered after surgical treatment of said animal for said cancer.

26. The method of claim 1, wherein said disorder is an autoimmune disease.

27. (canceled)

28. The method of claim 1, wherein said disorder is rheumatoid arthritis.

29. The method of claim 1, wherein said disorder is an inflammatory disease.

30. The method of claim 1, wherein said disorder is a skin disease.

31. The method of claim 1, wherein said disorder is psoriasis.

32. (canceled)

33. A compound having the Formula II: 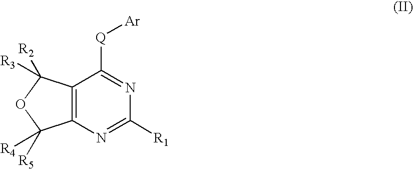
or a pharmaceutically acceptable salt or prodrug or tautomer thereof, wherein:
Ar is optionally substituted aryl or optionally substituted heteroaryl;
Q is O, S or NR₆, wherein R₆ is hydrogen or an optionally substituted alkyl;
R₁ is hydrogen, halo, optionally substituted amino, alkylamino, arylamino, alkylthiol, alkylsulfone, alkylsulfoxide, arylthiol, alkoxy, or aryloxy, optionally substituted C₁₋₁₀ alkyl, haloalkyl, aryl, carbocyclic, a heterocyclic group, a heteroaryl group, alkenyl, alkynyl, arylalkyl, arylalkenyl, arylalkynyl, heteroarylalkyl, heteroarylalkenyl, heteroarylalkynyl, carbocycloalkyl, heterocycloalkyl, hydroxyalkyl, aminoalkyl, aminoaryl, carboxyalkyl, nitro, cyano, acylamido, hydroxy, acyloxy, azido, carboxy, or carbonylamido; and
R₂-R₅ independently are hydrogen, halo, amino, alkoxy, C₁₋₁₀ alkyl, haloalkyl, aryl, carbocyclic, a heterocyclic group, a heteroaryl group, alkenyl, alkynyl, arylalkyl, arylalkenyl, arylalkynyl, heteroarylalkyl, heteroarylalkenyl, heteroarylalkynyl, carbocycloalkyl, heterocycloalkyl, hydroxyalkyl, aminoalkyl, carboxyalkyl, nitro, cyano, acylamido, hydroxy, thiol, acyloxy, azido, carboxy, methylenedioxy, carbonylamido or alkylthiol, alkylsulfone, alkylsulfoxide.

34. The compound of claim 33, wherein Ar is optionally substituted pyrrazol, triaziol, thiadiazol, imidazole, oxazol, thiazol, phenyl, pyridyl, or pyrimidyl.

35. The compound of claim 33, wherein R₁ is hydrogen, halo, optionally substituted amino, alkylamino, or arylamino, optionally substituted alkoxy, aryloxy, optionally substituted alkylthiol, alkylsulfone, alkylsulfoxide, or arylthiol, optionally substituted aryl, optionally substituted heteroaryl, or optionally substituted C₁₋₁₀ alkyl.

36. The compound of claim 33, wherein Q is NH.

37. A compound of claim 33, wherein said compound is selected from the group consisting of:
5,7-Dihydro-N-(3,5-dimethoxyphenyl)-2-(methylthio)furo[3,4-d]pyrimidin-4-amine;
5,7-Dihydro-N-(4-methoxyphenyl)-N-methyl-2-(methylthio)furo[3,4-d]pyrimidin-4-amine;
5,7-Dihydro-N-(3,5-dimethoxyphenyl)-furo[3,4-d]pyrimidin-4-amine;
5,7-Dihydro-N-(3-bromophenyl)-2-methylfuro[3,4-d]pyrimidin-4-amine;
5,7-Dihydro-N-(3,5-dimethoxyphenyl)-2-methylfuro[3,4-d]pyrimidin-4-amine;
5,7-Dihydro-N-(3-methyl-1H-pyrazol-5-yl)-2-(methylthio)furo[3,4-d]pyrimidin-4-amine;
5,7-Dihydro-N-(3-methyl-1H-pyrazol-5-yl)-2-(methylsulfonyl)furo[3,4-d]pyrimidin-4-amine;
N-(4-(4-(3-Methyl-1H-pyrazol-5-ylamino)-5,7-dihydrofuro[3,4-d]pyrimidin-2-ylthio)phenyl)cyclopropanecarboxamide;
N-(4-(5,7-Dihydro-2-(methylsulfonyl)furo[3,4-d]pyrimidin-4-ylthio)phenyl)cyclopropanecarboxamide;
N-(4-(2-(3-Methyl-1H-pyrazol-5-ylamino)-5,7-dihydrofuro[3,4-d]pyrimidin-4-ylthio)phenyl)cyclopropanecarboxamide; and
5,7-Dihydro-N-(3-methyl-1H-pyrazol-5-yl)-2-(4-methylpiperazin-1-yl)furo[3,4-d]pyrimidin-4-amine;
or a pharmaceutically acceptable salt or prodrug thereof.

38-46. (canceled)

47. A compound having the Formula V: 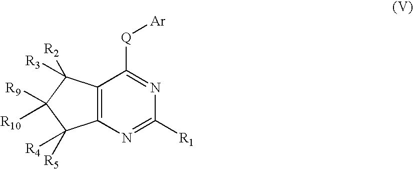
or a pharmaceutically acceptable salt or prodrug or tautomer thereof, wherein:
Ar is optionally substituted aryl or optionally substituted heteroaryl;
Q is O, S or NR₆, wherein R₆ is hydrogen or an optionally substituted alkyl;
R₁ is hydrogen, halo, optionally substituted amino, alkylamino, arylamino, alkylthiol, alkylsulfone, alkylsulfoxide, arylthiol, alkoxy, or aryloxy, optionally substituted C₁₋₁₀ alkyl, haloalkyl, aryl, carbocyclic, a heterocyclic group, a heteroaryl group, alkenyl, alkynyl, arylalkyl, arylalkenyl, arylalkynyl, heteroarylalkyl, heteroarylalkenyl, heteroarylalkynyl, carbocycloalkyl, heterocycloalkyl, hydroxyalkyl, aminoalkyl, aminoaryl, carboxyalkyl, nitro, cyano, acylamido, hydroxy, acyloxy, azido, carboxy, or carbonylamido; and
R₂-R₅ and R₉-R₁₀ independently are hydrogen, halo, amino, alkoxy, C₁₋₁₀ alkyl, haloalkyl, aryl, carbocyclic, a heterocyclic group, a heteroaryl group, alkenyl, alkynyl, arylalkyl, arylalkenyl, arylalkynyl, heteroarylalkyl, heteroarylalkenyl, heteroarylalkynyl, carbocycloalkyl, heterocycloalkyl, hydroxyalkyl, aminoalkyl, carboxyalkyl, nitro, cyano, acylamido, hydroxy, thiol, acyloxy, azido, carboxy, methylenedioxy, carbonylamido or alkylthiol, alkylsulfone, alkylsulfoxide.

48. The compound of claim 47, wherein Ar is optionally substituted pyrrazol, triaziol, thiadiazol, imidazole, oxazol, thiazol, phenyl, pyridyl, or pyrimidyl.

49. The compound of claim 47, wherein R₁ is hydrogen, halo, optionally substituted amino, alkylamino, or arylamino, optionally substituted alkoxy, aryloxy, optionally substituted alkylthiol, alkylsulfone, alkylsulfoxide, or arylthiol, optionally substituted aryl, optionally substituted heteroaryl, or optionally substituted C₁₋₁₀ alkyl.

50. The compound of claim 47, wherein Q is NR₆, and R₆ is hydrogen or an optionally substituted alkyl.

51. The compound of claim 47, wherein said compound is selected from the group consisting of:
6,7-Dihydro-N-(2,5-dimethoxyphenyl)-2-(methylthio)-5H-cyclopenta[d]pyrimidin-4-amine;
N-(3-Bromophenyl)-6,7-dihydro-2-(methylthio)-5H-cyclopenta[d]pyrimidin-4-amine;
6,7-Dihydro-N-(5-methyl-1H-pyrazol-3-yl)-2-(methylthio)-5H-cyclopenta[d]pyrimidin-4-amine;
6,7-Dihydro-N-(3,5-dimethoxyphenyl)-2-(methylthio)-5H-cyclopenta[c]pyrimidin-4-amine;
N-(4-(6,7-Dihydro-2-(methylthio)-5H-cyclopenta[d]pyrimidin-4-ylamino)phenyl)benzamide;
6,7-Dihydro-N-(4-methoxyphenyl)-N-methyl-2-(methylthio)-5H-cyclopenta[d]pyrimidin-4-amine;
6,7-Dihydro-N-(3,5-dimethoxyphenyl)-5H-cyclopenta[d]pyrimidin-4-amine;
6,7-Dihydro-N-(2,5-dimethoxyphenyl)-5H-cyclopenta[d]pyrimidin-4-amine;
N-(4-(6,7-Dihydro-5H-cyclopenta[d]pyrimidin-4-ylamino)phenyl)benzamide;
6,7-Dihydro-N-(3,5-dimethoxyphenyl)-2-(methylsulfinyl)-5H-cyclopenta[d]pyrimidin-4 amine;
or a pharmaceutically acceptable salt or prodrug thereof.

52. A pharmaceutical composition comprising a pharmaceutically acceptable carrier and the compound of claim 33.

53. The pharmaceutical composition of claim 52, further comprising at least one known cancer chemotherapeutic agent, or a pharmaceutically acceptable salt of said agent.

54. The pharmaceutical composition of claim 52, further comprising at least one compound selected from the group consisting of busulfan, cis-platin, mitomycin C, carboplatin, colchicine, vinblastine, paclitaxel, docetaxel, camptothecin, topotecan, doxorubicin, etoposide, 5-azacytidine, 5-fluorouracil, methotrexate, 5-fluoro-2′-deoxy-uridine, ara-C, hydroxyurea, thioguanine, melphalan, chlorambucil, cyclophosamide, ifosfamide, vincristine, mitoguazone, epirubicin, aclarubicin, bleomycin, mitoxantrone, elliptinium, fludarabine, octreotide, retinoic acid, tamoxifen, Herceptin®, Rituxan®, arsenic trioxide, gemcitabine, doxazosin, terazosin, tamsulosin, CB-64D, CB-184, haloperidol, lovastatin, simvastatin, pravastatin, fluvastatin, atorvastatin, cerivastatin, amprenavir, abacavir, CGP-73547, CGP-61755, DMP-450, indinavir, nelfinavir, tipranavir, ritonavir, saquinavir, ABT-378, AG1776, BMS-232,632, bexarotene, tretinoin, 13-cis-retinoic acid, 9-cis-retinoic acid, α-difluoromethylornithine, ILX23-7553, fenretinide, N-4-carboxyphenyl retinamide, lactacystin, MG-132, PS-341, Gleevec®, ZD1839 (Iressa), SH268, genistein, CEP2563, SU6668, SU11248, EMD121974, R115777, SCH66336, L-778,123, BAL9611, TAN-1813, flavopiridol, UCN-01, roscovitine, olomoucine, celecoxib, valecoxib, rofecoxib and alanosine.
